(19) United States
(12) Patent Application Publication
(10) Pub. No.: US 2026/0124407 A1
(43) Pub. Date: May 7, 2026

(54) SURGICAL FLUID DELIVERY SYSTEM AND COMPONENTS THEREOF

(71) Applicant: Fisher & Paykel Healthcare Limited, Auckland (NZ)

(72) Inventors: Richard John BOYES, Auckland (NZ); Benjamin Elliot Hardinge PEGMAN, Auckland (NZ); Rochelle Lynne Briones BIBAL, Auckland (NZ); Alice Chall-Kei NG, Auckland (NZ); Zane Paul GELL, Auckland (NZ); Christopher Keith WATSON, Auckland (NZ); Xueyan BIAN, Auckland (NZ)

(21) Appl. No.: 19/101,041

(22) PCT Filed: Aug. 29, 2023
(86) PCT No.: PCT/IB2023/058510

§ 371 Date: Feb. 4, 2025

Related U.S. Application Data
(60) Provisional application No. 63/373,917, filed on Aug. 30, 2022.

(51) Int. Cl. A61M 13/00; A61B 1/00; A61B 1/313

(52) U.S. Cl. CPC A61M 13/003; A61B 1/00154; A61B 1/3132; A61M 2202/0007; A61M 2202/02

(57) ABSTRACT

A medical instrument accessory for providing fluid to a body cavity of a patient during a surgical procedure. The medical instrument accessory comprises a body mountable over at least a portion of a shaft of a medical instrument. The body comprises an inner wall defining a lumen, a proximal end; a distal end defining an opening; a fluid connecting portion configured to connect the lumen in fluid connection with a fluid source; and a sealing element configured to provide a substantially fluid-tight seal with the medical instrument. The sealing element is configured to allow rotation of the medical instrument accessory relative to the medical instrument shaft, such that rotational movement of the fluid connecting portion is decoupled from rotational movement of the medical instrument shaft. Also disclosed are: a swivel connector for a medical instrument accessory; a flow controller for controlling flow in a fluid evacuation line for a surgical system; a fluid line connector for joining a first fluid evacuation line and a second fluid evacuation line to a suction canister; a fluid evacuation system; and a system for providing fluid to and venting fluid from a body cavity of a patient during a surgical procedure.

### FIELD OF THE DISCLOSURE

The present disclosure relates to systems and components of systems configured to deliver fluid to and/or vent fluid from a body cavity of a patient, in particular during a medical procedure.

### BACKGROUND

Various medical procedures may require the provision of fluid, such as carbon dioxide gas, to a patient during the procedure. Medical procedures may additionally or alternatively require venting of fluid from the body cavity.

An insufflator may be arranged to deliver fluid to a body cavity of the patient to inflate the body cavity and/or to resist collapse of the body cavity during a surgical procedure. Examples of such medical procedures include laparoscopy and endoscopy, although an insufflator may be used with any other type of medical procedure as required. Endoscopic procedures enable a medical practitioner to visualize a body cavity by inserting an endoscope, or the like, through one or more natural openings, small puncture(s), or incision(s) to generate an image of the body cavity. In laparoscopy procedures, a medical practitioner typically inserts a medical instrument through natural openings, small puncture(s), or incision(s) to perform a medical procedure in the body cavity. In some cases an initial endoscopic procedure may be carried out to assess the body cavity, and then a subsequent laparoscopy carried out to operate on the body cavity. Such procedures are widely used, for example, on the peritoneal cavity, or during a thoracoscopy, colonoscopy, gastroscopy or bronchoscopy.

Vision through a viewing portion (e.g. lens) of the instrument can be impaired by a number of factors. For example, vision may be impaired when condensation forms on the viewing portion, and/or when condensation forms on the instrument, which can coalesce into water droplets and drip onto the viewing portion. Vision may also be impaired by smoke in the cavity (for example, from cauterisation or similar) and/or smoke particles adhering to the viewing portion.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure provides examples of a system configured to deliver fluid to and/or vent fluid from a body cavity of a patient during a medical procedure, for example a surgical procedure. The present disclosure also provides examples of systems, and components of systems, configured to deliver fluid to and/or vent fluid from a body cavity of a patient during a medical procedure. The system components may include one or more of a medical instrument accessory, a flow controller and a fluid line connector.

Condensation may occur when the temperature of a gas falls below the dew point temperature for the level of humidity the gas is carrying. This may be caused by the gas contacting a surface which is at a temperature below the dew-point temperature of the gas. The human body is a warm and humid environment and can have a temperature (such as a core body temperature, for example) of about 37° C. Prior to being inserted into this environment, medical instruments intended for insertion into the body cavity, such as cameras, scopes, or other medical instruments may have a temperature, and/or can be exposed to temperatures, at or below typical room temperature and/or below a typical human body temperature. When inserted into this environment, condensation or fogging can form on a viewing portion of the medical instrument (such as a scope) or elsewhere on the medical instrument. In some cases, this condensation can coalesce into droplets. Condensation can also form elsewhere, such as within a cannula through which the medical instrument is inserted, which can coalesce into droplets and migrate down towards, and onto, the viewing portion. Further, when the instrument is removed from the body cavity, this may cause the temperature of the instrument to decrease, which can result in further fogging and/or condensation when reintroduced to the cavity. Condensation, fogging, and/or droplets on the viewing portion can impair vision of a user of the medical instrument.

Additionally and/or alternatively, during a medical procedure, various other substances can contact the viewing portion of the medical instrument, and/or come within view of the viewing portion of the medical instrument, which can impair vision. For example, the viewing portion of a scope may contact or be positioned near particles created by the procedure, such as surgical smoke. When the viewing portion becomes contaminated by particles, fluid droplets, or the like, it may be necessary to remove the medical instrument and clean the visual impairment. However, removing a medical instrument from the body cavity can cause it to cool, for example below the patient's body temperature, such that when the instrument is reinserted to the body, further condensation and/or droplets can form which, again, can inhibit vision through the viewing portion. This process may need to be repeated multiple times. Past approaches to resolve this include pre-warming the medical instruments, and/or using a light or a heating source at the end of the camera to warm the viewing portion. Such interventions typically require additional steps that can negatively impact the workflow and efficiency of the procedure. Furthermore, repetitive heating of instruments, or parts thereof, such as with a heating element adjacent a viewing portion, can affect the structure of the instrument, and/or increase complexity of sterilizing the instrument.

The present disclosure provides examples of a medical instrument accessory configured to direct fluid relative to an end of the cannula/medical instrument. In particular, disclosed examples are suitable for directing fluid flow around and/or near a distal end of a medical instrument, and/or directing fluid flow towards a desired region of the body cavity. This can prevent or at least reduce condensation and/or droplets forming on within the cannula or the viewing portion of the medical instrument, and/or prevent or at least reduce other substances from contacting or coming within view of the viewing portion.

The medical instrument accessory may be connected to tubing for supply and/or venting of fluid. Such tubing may adversely impact manoeuvrability of the medical instrument in use. The present disclosure provides examples of a medical instrument accessory in which rotational movement of the medical instrument is decoupled from rotational movement of connected tubes. In the disclosed medical instrument examples, the medical instrument may be rotated while the connected tubing remains in its initial position. This may enhance manoeuvrability of the medical instrument and/or associated components such as the cannula.

The present disclosure provides examples of the medical instrument accessory in which relative rotational movement between the medical instrument and at least a portion of the medical instrument accessory and/or fluid connecting portion is permitted, while maintaining retention of the medical instrument accessory on the medical instrument and/or while maintaining a seal between the medical instrument accessory and the medical instrument.

Rotational decoupling of the fluid connecting portion may enable fluid tubing connected to the medical instrument accessory to remain in essentially same position/orientation during manipulation of the medical instrument, by virtue of weight of tube. The medical instrument may therefore be able to be manipulated as needed while minimising interference due to fluid tubing orientation.

The present disclosure provides examples of a sealing element of the medical instrument accessory. The sealing element can be configured to seal against a medical instrument which is inserted into the medical instrument accessory to prevent fluid (such as gas) from escaping from a proximal end of the accessory and/or retains the medical instrument in position within the medical instrument accessory. The sealing element can be configured to provide a first resistance upon insertion of the medical instrument into the medical instrument accessory, and a second resistance upon removal of the medical instrument from the medical instrument accessory.

The sealing element can be configured to allow the medical instrument to be rotated within the medical instrument accessory, and/or the medical instrument accessory to be rotated about the medical instrument. For example, the sealing element may be configured to allow a shaft of the medical instrument to rotate within the seal, and/or the seal to rotate about the medical instrument.

Additionally, or alternatively, the sealing element can be configured as a securing element, to hold or otherwise retain the medical instrument accessory on the medical instrument shaft in at least one desired position.

According to one disclosed aspect, there is provided a medical instrument accessory for providing fluid to and/or from a body cavity of a patient during a surgical procedure, the medical instrument accessory comprising:
a body mountable over at least a portion of a shaft of a medical instrument, the body comprising:
   an inner wall defining a lumen,
   a proximal end;
   a distal end defining an opening; and
   a fluid connecting portion configured to connect the lumen in fluid connection with a fluid source,
   a sealing element configured to provide a substantially fluid-tight seal with the medical instrument;
wherein the body is configured such that rotational movement of the fluid connecting portion is decoupled from rotational movement of the medical instrument.

According to one disclosed aspect, there is provided a medical instrument accessory for providing fluid to and/or from a body cavity of a patient during a surgical procedure, the medical instrument accessory comprising:
a body mountable over at least a portion of a shaft of a medical instrument, the body comprising:
   an inner wall defining a lumen,
   a proximal end;
   a distal end defining an opening; and
   a fluid connecting portion configured to connect the lumen in fluid connection with a fluid source,
   a sealing element configured to provide a substantially fluid-tight with the medical instrument;
wherein the sealing element is configured to allow rotation of the medical instrument accessory relative to the medical instrument shaft, such that rotational movement of the fluid connecting portion is decoupled from rotational movement of the medical instrument shaft.

According to one disclosed aspect, there is provided a medical instrument accessory for providing fluid to and/or from a body cavity of a patient during a surgical procedure, the medical instrument accessory comprising:
a body mountable over at least a portion of a shaft of a medical instrument, the body comprising:
   an inner wall defining a lumen,
   a proximal end;
   a distal end defining an opening;
   a fluid connecting portion configured to connect the lumen in fluid connection with a fluid source;
   a sealing element configured to provide a substantially fluid-tight seal with the medical instrument; and
   a securing element configured to maintain a position of the medical instrument shaft relative to at least a portion of the medical instrument accessory;
wherein the body is configured such that rotational movement of the fluid connecting portion is decoupled from rotational movement of the sealing element and/or the securing element.

The medical instrument accessory described in the preceding paragraphs may further comprise one or more of the following features.

The sealing element may be configured to attach to the medical instrument shaft. The sealing element may be configured to allow rotation of the medical instrument shaft relative to the seal. Rotation of the medical instrument shaft relative to the seal may enable rotation of the fluid connecting portion about a longitudinal axis of the medical instrument shaft. The fluid-tight seal may be substantially maintained during relative rotation between the medical instrument shaft and the medical instrument accessory. The seal may provide a fluid-tight seal with the shaft of the medical instrument.

The sealing element may be configured to retain the medical instrument in position, for example in a desired longitudinal and/or rotational position, within the medical instrument accessory. A resistance to movement between the sealing element and the medical instrument shaft may be configured to allow relative rotational movement between the sealing element and the medical instrument shaft.

The sealing element may be provided at or adjacent to a proximal end of the lumen. The sealing element may be configured to attach at or adjacent to a proximal end of the medical instrument shaft.

The body may be configured to receive the medical instrument shaft by sliding insertion of the medical instrument shaft to the lumen. The body may comprise a guide portion at the proximal end to guide the medical instrument shaft during insertion.

According to one disclosed aspect, there is provided a medical instrument accessory for providing fluid to a body cavity of a patient during a surgical procedure, the medical instrument accessory comprising:
a body mountable over at least a portion of a shaft of a medical instrument, the body comprising:
   an inner wall defining a lumen,
   a proximal end;
   a distal end defining an opening; and
   a fluid connecting portion configured to connect the lumen in fluid connection with a fluid source,
   a sealing element configured to provide a substantially fluid-tight seal with the medical instrument;
   wherein the sealing element is further configured to provide a resistance to movement of the medical instrument shaft relative to the sealing element.

In some examples, the resistance may comprise a resistance to longitudinal movement of the shaft relative to the sealing element. The resistance may be configured to retain the medical instrument in at least one desired position within the medical instrument accessory. The desired position may be a desired longitudinal position and/or a desired rotational position relative to the medical instrument shaft.

The body may be configured to receive the medical instrument shaft by sliding insertion of the medical instrument shaft to the lumen. The sealing element may be configured to provide a first resistance upon insertion of the medical instrument into the lumen and a second resistance upon withdrawal of the medical instrument shaft from the lumen. The second resistance may be greater than the first resistance.

The sealing element may be configured to allow the medical instrument to be rotated within the medical instrument accessory. For example, the sealing element may retain the medical instrument accessory in desired longitudinal position whilst allowing rotation of the medical instrument within the seal. In other examples, the sealing element may be configured to inhibit the medical instrument from rotating relative to the sealing element.

Configuring the sealing element to provide a resistance may include configuring a width of the sealing element. Configuring the sealing element to provide a resistance may include configuring a surface area of a contact surface in contact with the outer wall of the medical instrument shaft. Configuring the sealing element to provide a resistance may include selecting material properties of the seal.

The sealing element may be at least partially formed from a material which is one or more of flexible, deformable, resilient and/or compressible.

Movement of the medical instrument shaft in a proximal direction may deform the sealing element in a proximal direction. The body may comprise a seal retaining structure configured to limit deformation of the sealing element in at least the proximal direction. The seal retaining structure may be configured as an abutment surface extending radially inward from the inner surface of the lumen, and wherein sliding movement of the medical instrument shaft in a proximal direction compresses at least a portion of the sealing element against the abutment surface.

Configuring the sealing element to provide a resistance may include treating a surface of the sealing element to modify frictional properties of the surface. In some examples, the surface may be treated to reduce friction. For example, the surface may be textured to reduce friction between the surface and the medical instrument. The surface may be textured to increase surface roughness to reduce friction. The surface may be treated by, for example, sandblasting and/or by polishing. The surface may be treated by application of a low-friction layer or coating.

The surface treated to reduce friction may be a surface configured to contact the medical instrument during insertion of the medical instrument to the medical instrument accessory. Treatment of the surface to reduce friction may facilitate a relatively reduced force required to insert the medical instrument through the seal, compared to a force required to withdraw the medical instrument from the seal.

In some examples, the sealing element may comprise a proximal surface. The proximal surface may be tapered. The tapered proximal surface may be configured to reduce friction on the medical instrument shaft during insertion to the lumen.

In some examples, an inwardly facing portion of the sealing element may be substantially devoid of corners or sharp transitions in curvature.

In some examples, the sealing element may comprise a distal surface. The distal surface may be tapered.

The medical instrument accessory may comprise one or more securing elements configured to hold or otherwise retain the medical instrument accessory on the medical instrument shaft in the desired position(s). In such examples, the medical instrument accessory may be configured such that the fluid connecting portion is rotationally decoupled from the securing element.

In some examples, the sealing element may function as the securing element. That is, the sealing element and securing element may be the same component. Additionally, or alternatively, the medical instrument accessory may comprise one or more non-sealing securing elements.

The securing element may include a non-sealing structure. In some examples, the securing element may include a locking mechanism operable to secure an accessory for the medical instrument to the medical instrument.

The securing element may be arranged adjacent an opening defined at a proximal end of the medical instrument accessory.

The securing element may include a clamp, collar, or other similar structure, which may be positioned adjacent the proximal end opening of the medical instrument accessory and tightened to hold the accessory on the medical instrument.

The locking mechanism may include a cam. The cam may be rotatable about an axis to move the cam between a first (open) position and a second (locking) position. Arranging the cam in the locked position may allow the cam to interfere with the medical instrument to secure the medical instrument shaft in the medical instrument accessory. The locking mechanism may include a lever extending from the cam. The locking mechanism may be configured to be operable by a user operating the lever.

In some examples, the body may comprise a guide portion at the proximal end to guide the medical instrument shaft during insertion to the lumen.

According to one disclosed aspect, there is provided a medical instrument accessory for providing fluid to a body cavity of a patient during a surgical procedure, the medical instrument accessory comprising:
a body mountable over at least a portion of a shaft of a medical instrument, the body comprising:
   an inner wall defining a lumen,
   a proximal end;
   a distal end defining an opening;
   a first portion comprising a sealing element configured to provide a substantially fluid-tight seal with the medical instrument; and
   a second portion comprising a fluid connecting portion configured to connect the lumen in fluid connection with a fluid source,
   wherein the second portion is rotatably coupled to the first portion. The rotatable coupling of the first portion and second portion may be configured such that rotational movement of the fluid connecting portion is decoupled from rotational movement of the medical instrument shaft.

The medical instrument accessory may be configured such that the fluid connecting portion is rotatable (about a longitudinal axis of the accessory) independently of the seal element and/or one or more other portion(s) of the medical instrument accessory.

The second portion may be rotatable relative to the first portion about a longitudinal axis of the body. The sealing element and/or the securing element may be fixed relative to the first portion.

The first and second portions may be configured to be connected together. The first and second portions may be configured to be rotatable when connected to each other. The first and second portions may comprise respective coupling elements configured to interlock, such that the first and second portions are axially fixed but allowed to rotate relative to each other. The interlocking connection may be configured to be a permanent engagement.

The connection between the first and second portions may permit rotation of the fluid connecting portion relative to one or more other portions of the medical instrument accessory. The connection between the first and second portions may be positioned distally of the sealing element.

The coupling elements may comprise respective mating surfaces. The mating surfaces may be configured to mate to inhibit leakage of fluid. In some examples, the mating surfaces may mate to provide a substantially fluid-tight seal.

In some examples, one of the coupling elements may comprise a male swivel connector portion while the other of the coupling elements comprises a female swivel connector portion. The first portion may be configured to partially receive the second portion (or the second portion may be configured to partially receive the first portion).

The coupling elements may comprise a projection and a circumferential groove for receiving the projection. The coupling elements may be configured to interlock via a suitable connection, such as, for example, snap-fit connection, press/friction fit, welding, threaded engagement and/or adhesive.

The coupling elements may be configured to minimise friction between the mating surfaces, to promote free rotation between the first and second portions of the body. For example, the coupling elements may be formed from a plastics material having low friction properties.

The first portion may be a first conduit component. The second portion may be a second conduit component.

According to one aspect of the present disclosure, there is provided a swivel connector for a medical instrument accessory for providing fluid to a body cavity of a patient during a surgical procedure, the medical instrument accessory comprising:
a first conduit component; and
a second conduit component;
   wherein the first conduit component comprises a sealing element and/or a securing element, wherein the first conduit component and the second conduit component are configured to be connected together, and wherein, when connected, the first conduit component and the second conduit component are configured to be rotatable relative to one another.

The second conduit component may be configured to receive at least part of the first conduit component. The first conduit component may be configured to receive at least part of the second conduit component.

In some examples, the body may further comprise a third portion, rotatably coupled to the second portion. The second portion may be located between the first portion and the third portion. The third portion may be rotationally fixed relative to the first portion.

In any of the above-described aspects, the medical instrument accessory may further comprise at least one guide element on or within the inner wall of the accessory. The at least one guide element may be configured to position the shaft of the medical instrument within the lumen.

In some examples, the at least one guide element may hold the medical instrument shaft substantially concentrically within the lumen. The at least one guide element may comprise one or more projections (for example, ribs, bumps, fins and/or splines) extending inwardly from the wall.

In some examples, the at least one guide element may comprise one or more ribs extending inwardly from the inner wall. The one or more ribs may be located at or adjacent to the distal end of the lumen.

Additionally and/or alternatively, the at least one guide element may comprise one or more channels, notches, dimples and/or grooves within the inner wall. The at least one guide element can be located at the proximal end, the distal end and/or be intermediate along the length of the lumen. In some examples, the at least one guide element comprises a plurality of guide elements. The guide elements can be spaced substantially uniformly around the lumen. The guide elements can be spaced non-uniformly around the lumen.

According to one disclosed aspect, there is provided a system for providing fluid to a body cavity of a patient during a surgical procedure, the system including:
a medical instrument accessory according to the present disclosure; and
one or more fluid delivery tubes for connecting to the fluid connecting portion of the medical instrument accessory.

The system may further comprise one or more filters. The system may further comprise one or more cannulas.

According to one disclosed aspect, there is provided a system for providing fluid to a body cavity of a patient during a surgical procedure, the system including:
a medical instrument accessory according to the present disclosure; and
one or more cannulas.

In some examples, at least one cannula may be configured to supply fluid to the body cavity. In some examples, at least one cannula may be configured to vent fluid from the body cavity. In some examples, at least one cannula may be configured to supply fluid to the body cavity and vent fluid from the body cavity simultaneously.

The system may further comprise one or more filters. The system may further comprise one or more fluid delivery tubes for connecting to the fluid connecting portion of the medical instrument accessory.

According to one disclosed aspect, there is provided a flow controller for controlling flow in a fluid evacuation line for a surgical system, the flow controller comprising:
a body comprising:
   a first body portion configured for fluid communication with a first portion of the fluid evacuation line;
   a first opening in fluid communication with the first portion of the fluid evacuation line;
   a second body portion coupled to the first body portion, the second body portion configured for connection with a second portion of the fluid evacuation line; and
   a second opening in fluid communication with the second portion of the fluid evacuation line;
wherein the first and second body portions are movable relative to each other to bring the first opening into or out of fluid communication with the second opening, thereby to selectively open, close and/or vary a fluid flow path between the first and second portions of the fluid evacuation line.

According to one disclosed aspect, there is provided a flow controller for controlling flow in a fluid evacuation line for a surgical system, the flow controller comprising:
a body comprising:
   a first body portion comprising a first fluid connecting portion configured for connection with a first portion of the fluid evacuation line, the first body portion defining a first opening in fluid communication with the first fluid connecting portion; and
   a second body portion coupled to the first body portion, the second body portion comprising a second fluid connecting portion configured for connection with a second portion of the fluid evacuation line, the second body portion defining a second opening in fluid communication with the second fluid connecting portion,
wherein the second body portion is movable relative to the first body portion to bring the first opening into or out of fluid communication with the second opening, thereby to selectively open, close and/or vary a fluid flow path between the first fluid connecting portion and the second fluid connecting portion.

The second body portion may be movable relative to the first body portion from a first position in which the first opening and the second opening are out of alignment relative to each other and the fluid flow path is closed, to a second position in which the first opening and the second opening are in at least partial alignment relative to each other and the fluid flow path is at least partially open.

The first fluid connecting portion may comprise a fluid inlet and the second fluid connecting portion may comprise a fluid outlet.

The first and second body portions may comprise respective walls. The wall of the first body portion may comprise the first opening. The wall of the second body portion may comprise the second opening.

The walls of the first and second body portions may be slidably engaged with each other. For example, the walls of the first and second body portions may be axially and/or rotationally slidably engaged with each other.

In the first position, the first opening may be obstructed by the wall of the second body portion to inhibit fluid flow therethrough. In the second position, the first opening may be substantially (or at least partially) unobstructed to allow fluid flow therethrough. In at least one intermediate position between the first position and the second position, the first opening may be partially obstructed by the wall of the second body portion to partially restrict fluid flow therethrough.

The wall of the second body portion may define an inner portion of the second body portion. The inner portion may be in fluid communication with the second fluid connecting portion. At least a portion of the inner portion of the second body portion may be received (for example, nested) within the first body portion.

The walls of the first and second body portions may include respective cylindrical side-walls. At least a portion of one cylindrical side-wall may be received within the other cylindrical side-wall (for example, in a nested arrangement). For example, the cylindrical side-wall of the second body portion may be at least partially received within the cylindrical side wall of the first body portion, or vice versa.

The first opening may extend through the wall (e.g. the cylindrical side wall) of the first body portion. The second opening may extend through the wall (e.g. the cylindrical side wall) of the second body portion.

Additionally or alternatively, the walls of the first and/or the second body portions may include respective end-walls. In some examples, the first opening may extend at least partially through the end-wall of the first body portion. The second opening may extend at least partially through the end-wall of the second body portion.

In some examples, an area of the first opening that is obstructed by the wall of the second body portion may vary as the second body portion is moved between the first position and the second position, to vary a flow rate through the fluid flow path. The opposite arrangement of first and second openings is also contemplated. That is, an area of the second opening that is obstructed by the wall of the first body portion may vary as the first body portion is moved between the first position and the second position, to vary a flow rate through the fluid flow path. As such, any of the features of either the first and/or second openings described herein may be applicable to the other of the first and/or second openings.

For example, the second opening may have a length and a height, wherein the height of the second opening varies along its length. For example, the second opening may taper along its length. In some examples, the second opening may have a shape which is substantially triangular.

In some examples, the second opening may be defined by a notch at one end of the wall of the second body portion.

The notch may include a ramped base portion. The notch may adjoin an open end of the inner cavity of the second body portion.

In some examples, a dimension of the first opening is shorter than the length of the second opening. The flow rate may be variable depending on a region of the second opening with which the first opening is aligned. The flow rate may be variable between a minimum flow rate of about 0 litres per minute and a maximum flow rate above 0, for example of about 7 litres per minute, about 10 litres per minute, about 12 litres per minute, about 15 litres per minute, about 20 litres per minute, or more.

The flow controller may comprise a movement limiter configured to limit the relative movement between the second body portion and the first body portion. The movement limiter may be configured to limit the relative movement to between limits defined at the first position and the second position.

The movement limiter may comprise a protrusion on one of the first body portion or the second body portion and at least one stop on the other of the first body portion or the second body portion. The protrusion may be configured to abut the at least one stop in the first position and/or the second position to inhibit movement beyond said positions.

In some examples, the second body portion may be rotatable relative to the first body portion. As such, the first position may be a first angular position. The second position may be a second angular position.

The first body portion and the second body portion may be axially fixed relative to one another. The second body portion may be rotatable relative to the first body portion about a longitudinal axis of the body. The length of the second opening may extend in a rotational direction.

The flow controller may comprise at least one indicator for indicating a relative alignment of the first and second body portions and/or a relative alignment of the first and second openings. The at least one indicator may include visual, haptic, auditory and/or tactile indication to a user.

The flow controller may comprise a first indicator on the first body portion and a second indicator on the second body portion, wherein a relative alignment of the first and second indicators may provide a visual indication of a position of the first opening relative to the second opening. Additionally or alternatively, the flow controller may comprise a first indicator on the first body portion and a second indicator on the second body portion, wherein a relative alignment of the first and second indicators provides a visual indication of a flow rate through the fluid flow path.

The flow controller may comprise one or more grip elements for facilitating movement of the second body portion relative to the first body portion by a user.

The movement or adjustment of the first body portion and second body potion relative to each other may be manually operable.

For example, the grip element may comprise a pair of radially extending fins. The fins may be positioned opposite each other about the longitudinal axis. In other examples, the fins may not be opposite each other. In some examples, a position of at least one of the fins relative to an indicator on the first body portion provides a visual indication of the position of the first opening relative to the second opening.

The flow controller may comprise at least one retaining element. The at least one retaining element may be configured for retaining the first and second body portions in relative alignment in one or more predefined positions.

The at least one retaining element may be configured to provide frictional resistance to relative movement between the first and second body portions. The frictional resistance may be configured to be able to be overcome by manual application of force between the first and second body portions by a user.

The at least one retaining element may comprise a plurality of retaining elements configured to retain the first and second body portions in relative alignment at a corresponding plurality of positions.

The at least one retaining element may be configured to retain the first and second body portions in relative alignment in the first position and/or in the second position. The at least one retaining element may be configured to retain the first and second body portions in relative alignment in at least one intermediate position between the first position and in the second position.

In some examples, the at least one retaining element may comprise at least one protrusion and at least one recess, the protrusion receivable in the recess. The protrusion may engage the recess by snap-fitting engagement.

In some examples, the flow controller may comprise an intermediate portion. In some examples, the first body portion may comprise a housing and an intermediate portion. In some examples the second body portion may comprise a housing and an intermediate portion.

The intermediate portion may be rotationally and axially fixed relative to the housing.

In some examples, the intermediate portion may be separate from either of the first body portion and second body portion.

The intermediate portion may comprise a fluid flow opening. In some examples, the first body portion comprises the first fluid opening and the intermediate portion comprises the second fluid opening. In other examples, the intermediate portion comprises the first fluid opening and the second body portion comprises the second fluid opening.

The intermediate portion may be configured to be received in one of the first body portion and the second body portion. The intermediate portion may be configured to receive the other of the first body portion and the second body portion.

In some examples, the intermediate portion may comprise an insert of the first body portion, or of the second body portion. The first or second body portion body portion may comprise a housing and the insert. The first opening may be provided through the insert. The first fluid connecting portion may be provided on the housing.

The insert may be rotationally and axially fixed relative to the housing. For example, the insert may be configured for engagement with the housing to inhibit relative axial movement of the insert and the housing.

The first body portion, or second body portion, may comprise a rotation-inhibiting element for inhibiting relative rotation of the housing and the intermediate portion. The rotation-inhibiting element may comprise a slot on one of the housing or the intermediate portion and a protrusion on the other of the housing or the intermediate portion, the protrusion receivable in the slot.

The intermediate portion may comprise the wall of the first body portion. The wall of the first body portion may be located at least partially within an internal portion of the housing. The housing may define the internal portion in fluid connection with the first opening and the first fluid connecting portion.

In some examples, a narrowest passage of the fluid flow path is defined by the first opening and/or by the second opening. For example, the body of the flow controller may not include a filter.

The first body portion may be in axial alignment with the second body portion. The first fluid connecting portion may be in axial alignment with the second fluid connecting portion. The first fluid connecting portion and the second fluid connecting portion may be coincident with the longitudinal axis of the body.

According to one disclosed aspect, there is provided a fluid evacuation system for evacuating gas and irrigation fluid from a body cavity of a patient, the system comprising:
a first fluid evacuation line in fluid communication with the body cavity for evacuating the gas from the body cavity; and
a second fluid evacuation line in fluid communication with the body cavity for evacuating the irrigation fluid from the body cavity,
   wherein the first fluid evacuation line and the second fluid evacuation line are connected to a single connection port for connection to a suction canister.

The fluid evacuation may include a one-way valve in connection with the first fluid evacuation line, upstream of the connection with the second fluid evacuation line.

According to one disclosed aspect, there is provided a fluid line connector for joining a first fluid evacuation line and a second fluid evacuation line to a suction canister, the fluid line connector comprising:
a connector body comprising:
   a first inlet port for fluid connection with the first evacuation line;
   a second inlet port for fluid connection with the second fluid evacuation line;
   an outlet port for fluid connection with the suction canister; and
   a junction region fluidly connecting the first inlet port, the second inlet port and the outlet port,
   wherein a one-way valve is positioned within the connector body and configured to inhibit fluid flow from the junction region into the first inlet port.

The fluid evacuation system may include the fluid line connector.

The first inlet port and the second inlet port may be substantially perpendicular to each other. The first inlet port and/or the second inlet port may be perpendicular to the outlet port. The second inlet port and outlet may be axially aligned. In other examples, the first and second inlet port and outlet port may be positioned at other angles relative to each other.

The first inlet port may be positioned upstream of the outlet port. The one-way valve may be configured to be positioned upstream of the outlet port. The one-way valve may be configured to be positioned in or adjacent the first inlet port.

The first inlet port may be configured to be positioned, in use, above the second inlet port and/or above the outlet port. When in use, the one-way valve may be configured to be positioned above the second inlet port and/or above the outlet port. In other examples, inlets, outlet and valve may be configured for use in other relative orientations.

In some examples, the one-way valve may be positioned within the connector body. For example, the one-way valve may be positioned within the first inlet port. The one-way valve may be configured to inhibit fluid flow from the junction region into the first inlet port. In other examples, the one-way valve may be spaced from the connector body and configured to inhibit fluid flow from the junction region through the first fluid evacuation line past the location of the one-way valve. For example, the one-way valve may be connected to the connector body by a length of tubing. Additionally or alternatively, the one-way valve may be provided in a length of tubing connected to the connector body.

The fluid evacuation system may comprise at least one filter in fluid connection with the first fluid evacuation line. The filter may be configured to filter smoke and/or particulate matter contained in fluid flowing through the first fluid evacuation line.

The at least one filter may be positioned upstream of the one-way valve. In some examples, the fluid line connector may comprise a filter. For example, the at least one filter may be positioned within the connector body. Additionally or alternatively, at least one filter may be removably connectable to the connector body. The filter may be positioned upstream of the second inlet port and/or upstream of the outlet port.

According to one disclosed aspect, there is provided a system for providing fluid to and venting fluid from a body cavity of a patient during a surgical procedure, the system including:
a medical instrument accessory for providing fluid to the body cavity, the medical instrument accessory comprising:
   a body mountable over at least a portion of a shaft of the medical instrument, the body comprising:
   an inner wall defining a lumen,
   a proximal end;
   an open distal end;
   a fluid connecting portion configured to fluidly connect the lumen with a fluid source; and
   at least one structure configured to position the medical instrument shaft in the lumen such that a fluid flow path is defined between the lumen inner wall and the medical instrument shaft; and
a first fluid evacuation line configured for connection with an outlet port in fluid communication with the body cavity to vent fluid from the body cavity.

The at least one structure may be configured such that fluid can be directed into the open distal end or out from the open distal end and around an end of the medical instrument.

The system may further comprise a flow controller for controlling fluid flow in the first fluid evacuation line. The flow controller may be as described in any of the examples herein. The system may comprise one or more fluid tubes for connecting to the first fluid connecting portion and the second fluid connecting portion of the flow controller to form at least part of the first fluid evacuation line.

At least one fluid tube may be configured for connection to a suction source. At least one fluid tube is configured for connection to at least one patient interface. The at least one patient interface may include at least one cannula. The at least one cannula may be configured to facilitate venting of fluid from the body cavity.

The system may further comprise one or more filters. At least one filter may be configured to filter smoke and/or particulate matter from vented fluid.

The system may comprise a fluid evacuation system for joining the first fluid evacuation line and a second fluid evacuation line to a common suction canister. The fluid evacuation system may be the fluid evacuation system as described in examples herein.

The medical instrument accessory may comprise a sealing element configured to provide a substantially fluid-tight seal with the medical instrument. The sealing element may be configured to provide a substantially fluid-tight seal with the medical instrument shaft.

The medical instrument accessory may be configured such that the fluid connecting portion is rotatable (about a longitudinal axis of the accessory) independently of the seal element and/or independently of one or more other portion(s) of the medical instrument accessory.

The sealing element may be configured to allow rotation of the medical instrument accessory relative to the medical instrument shaft, such that rotational movement of the fluid connecting portion is decoupled from rotational movement of the medical instrument shaft. The sealing element may be configured to provide a resistance to movement of the medical instrument shaft relative to the sealing element.

In other examples, the sealing element may be configured to inhibit the medical instrument from rotating relative to the sealing element.

The body of the medical instrument accessory may include a first portion comprising the sealing element and/or a securing element and a second portion comprising the fluid connecting portion, wherein the first portion is rotatably decoupled from the second portion. The second portion may be rotatable relative to the first portion about a longitudinal axis of the body.

The sealing element and/or the securing element may be fixed relative to the first portion.

The medical instrument accessory may comprise:
a first portion comprising a sealing element configured to provide a substantially fluid-tight seal with the medical instrument; and
a second portion comprising a fluid connecting portion configured to connect the lumen in fluid connection with a fluid source,
wherein the second portion is rotatably coupled to the first portion.

The medical instrument accessory may comprise at least one guide element on or within the inner wall of the accessory configured to position the shaft of the medical instrument within the lumen. The at least one guide element may comprise one or more projections, for example, ribs, bumps, fins, pins, dimples in and/or on the inner wall. The at least one guide element may extend from the inner wall into the lumen.

The system may further comprise one or more fluid delivery tubes for connecting to the fluid connecting portion of the medical instrument accessory.

The system may, optionally, comprise a humidifier in fluid connection with at least one fluid delivery tube. In some examples, the system may not include a humidifier.

The system may comprise at least one filter in fluid connection with the outlet of the humidifier chamber, downstream of the outlet.

The system may comprise a suction canister. The suction canister may have a single inlet port.

It will be appreciated that references to “proximal” and “distal” in this specification are in accordance with the conventional meanings in the art for these words, where the terms are relative to an operator or user of a device. For example, a distal end of a medical instrument is typically the end arranged, in use, to be away from the operator, typically within or against the patient.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

These and other features, aspects, and advantages of the present disclosure are described with reference to the drawings of certain examples, which are intended to schematically illustrate certain examples and not to limit the scope of the disclosure. In some cases, a “slice” has been shown for clarity purposes for some sectional and cross-sectional views of a three-dimensional cannula, sheath, or accessory. A person skilled in the art would be able to appreciate that these figures illustrate a slice of a three-dimensional cannula, sheath, or accessory. In some cases, projecting surfaces have not been shown for clarity. For example, projecting hole surfaces are hidden in some views.

FIG. 1 illustrates, schematically, a medical gases delivery system according to one example of the present disclosure;

FIG. 2 illustrates, schematically, a medical gases delivery system in use in surgery according to one example of the present disclosure;

FIG. 3 illustrates a cross-section of a heated gases delivery tube of a medical gases delivery system according to one example of the present disclosure, suitable for use with the system of FIG. 1 or the system of FIG. 2;

FIG. 4 illustrates an example of a humidifier chamber suitable for use in the system of FIG. 1 or the system of FIG. 2;

FIG. 5 illustrates a body cavity without use of a medical instrument accessory according to the present disclosure;

FIG. 6 illustrates a medical instrument accessory according to one example of the present disclosure providing directed fluid flow;

FIG. 7 illustrates a medical instrument accessory according to one example of the present disclosure used as an exhaust/vent means;

FIGS. 8 to 14 illustrate a proximal end of a medical instrument accessory that can be attached to a medical instrument according to one example of the present disclosure;

FIGS. 15 and 16 illustrate a proximal end of a medical instrument accessory that can be attached to a medical instrument according to one example of the present disclosure;

FIGS. 17 to 23 illustrate various examples of a medical instrument accessory having a body including a rotatably coupled first portion and second portion;

FIGS. 24 and 25 illustrate directed fluid flow around a medical instrument according to one example of the present disclosure;

FIGS. 26 and 27 illustrate protrusions located on an inner surface of a medical instrument accessory according to one example of the present disclosure;

FIGS. 28 and 29 illustrate protrusions located at a distal end of the medical instrument accessory according to one example of the present disclosure;

FIGS. 30 and 31 illustrate protrusions located at a distal end of the medical instrument accessory according to one example of the present disclosure;

FIGS. 32 to 34 illustrates protrusions located at a first location at a distal end of the medical instrument accessory and a second spaced-apart location at the proximal end of the medical instrument accessory according to one example of the present disclosure;

FIGS. 35 and 36 illustrate uneven spacing of protrusions according to one example of the present disclosure;

FIGS. 37 and 38 illustrate protrusions of different widths according to one example of the present disclosure;

FIGS. 39 and 40 illustrate notches positioned at least partway along the body of a medical instrument accessory according to one example of the present disclosure;

FIGS. 41 and 42 illustrate a medical instrument accessory according to one example of the present disclosure with a cross-sectional shape that is non-circular thereby allowing fluid flow around the medical instrument;

FIG. 43 illustrates, schematically, a surgical system for providing fluid to and venting fluid from a body cavity of a patient during a surgical procedure according to one example of the present disclosure;

FIG. 44 is a perspective view of a flow controller according to one example of the present disclosure;

FIG. 45 is an exploded perspective view of the flow controller of FIG. 44;

FIG. 46 is a partially transparent view of a second body portion and insert of the flow controller of FIG. 44;

FIG. 47 illustrates, diagrammatically, relative alignments of a first and second opening of the flow controller in a first position (diagram A), a second position (diagram B) and a third position (diagram C);

FIG. 48 illustrates the second body portion of the flow controller of FIG. 44;

FIG. 49 illustrates a front view of the second body portion and the insert of the flow controller of FIG. 44, in a first position relative to each other;

FIG. 50 illustrates the second body portion of the flow controller of FIG. 44 in a position rotated relative to the position shown in FIG. 48;

FIG. 51 illustrates a front view of the second body portion and the insert of the flow controller of FIG. 44, in a second position relative to each other;

FIG. 52 illustrates a perspective view of the insert of the flow controller of FIG. 44;

FIG. 53 illustrates a perspective view of the insert of the flow controller of FIG. 44;

FIG. 54 illustrates the second body portion of the flow controller of FIG. 44;

FIG. 55 illustrates a partial, and partially transparent, perspective view of the second body portion and the insert of the flow controller of FIG. 44;

FIG. 56 illustrates a cross-sectional front view of a flow controller according to another example of the present disclosure having a first body portion and a second body portion;

FIG. 57 illustrates a cross-sectional side view of the flow controller of FIG. 56;

FIG. 58 illustrates a front view of the flow controller of FIG. 56;

FIG. 59 illustrates a side view of the flow controller of FIG. 56;

FIG. 60 illustrates a top view of a second body portion of a flow controller, according to another example of the present disclosure;

FIG. 61 illustrates a partial top view of an insert of a flow controller, according to another example of the present disclosure;

FIG. 62 illustrates a partial perspective view of the second body portion of FIG. 60;

FIG. 63 illustrates a perspective view of the insert of FIG. 61;

FIG. 64 illustrates, schematically, a prior art configuration for a system for fluid evacuation, including a first fluid evacuation line and a second fluid evacuation line in connection with respective suction canisters;

FIG. 65 illustrates, schematically, a fluid evacuation system according to one example of the present disclosure;

FIG. 66 is a front view of an example fluid line connector for use in the fluid evacuation system of FIG. 65;

FIG. 67 is a perspective view of the fluid line connector of FIG. 66;

FIG. 68 is another perspective view of the fluid line connector of FIG. 66;

FIG. 69 illustrates a cross-section of the fluid line connector of FIG. 66;

FIG. 70 illustrates a perspective cross-section of the fluid line connector of FIG. 65;

FIG. 71 illustrates a perspective view of a second body portion of a flow controller according to one example of the present disclosure;

FIG. 72 illustrates a perspective view of the second body portion of FIG. 71;

FIG. 73 illustrates a cross-section of a housing of a first body portion of a flow controller according to one example of the present disclosure;

FIG. 74 illustrates a cross-section of the housing of FIG. 73 and an insert of the first body portion according to one example of the present disclosure;

FIG. 75 illustrates a perspective view of the insert of FIG. 74;

FIG. 76 illustrates a perspective view of the housing of FIG. 73;

FIG. 77 illustrates a cross-section detail view of a rotatable connection of a medical instrument accessory according to one example of the present disclosure;

FIG. 78 illustrates a cross-section of a proximal region of a medical instrument accessory including the rotatable connection of FIG. 77;

FIG. 79 illustrates a cross-section of a proximal region of a medical instrument accessory according to another example of the present disclosure, including a rotatable connection having a reversed male-female configuration;

FIG. 80 illustrates a cross-section of a gas inlet of a medical instrument accessory according to one example of the present disclosure;

FIG. 81 illustrates a cross-section of a gas inlet of a medical instrument accessory according to another example of the present disclosure;

FIG. 82 illustrates a cross-section of a portion of a medical instrument accessory according to one example of the present disclosure, including a locking mechanism, with the locking mechanism in an open position;

FIG. 83 illustrates a cross-section of a portion of the medical instrument accessory of FIG. 82, with the locking mechanism in a closed position; and

FIG. 84 illustrates a proximal end of a medical instrument accessory that can be attached to a medical instrument according to one example of the present disclosure, including a flexible seal.

### DETAILED DESCRIPTION

Although certain examples are described below, it will be appreciated that the disclosure extends beyond the disclosed examples and/or uses, and includes obvious modifications and equivalents thereof. It is intended that the scope of the disclosure should not be limited by any particular examples described below. It will be appreciated that while some features may be disclosed in relation to one or more examples, and other features be disclosed in relation to one or more other examples, combining these features together in one or more further examples is within the scope of the disclosure. It would consequently be understood that any combinations of any disclosed features in an example of a medical instrument accessory, or the instrument itself, is within the scope of the disclosure.

### Examples of Surgical Systems and Medical Gases Delivery And/or Venting Systems

Systems for delivering fluid during medical procedures can include an insufflator, which may be operative to control the pressure and/or flow of the fluid from a fluid source to a level suitable for delivery into the body cavity. Fluid may be delivered via a cannula or needle connected to the system and inserted into the body cavity.

Such systems may be used in the context of minimally invasive surgeries, for example. Minimally invasive surgeries are performed by entering the body via small incisions through the skin, or via a natural body orifice. The body cavity (or surgical cavity) may include a viewing and/or working space for the surgery, sometimes referred to as the laparoscopic field.

It will be appreciated that a fluid as referred to herein may refer to any gas or liquid or a combination thereof. Where the term “gas” is used specifically, it should be understood that the system and/or components/devices described may also be suitable for use with other fluids.

It can be desirable to match the temperature of gases delivered from the system as closely as possible to the typical human body temperature. It can also be desirable to deliver gases above or below internal body temperature, such as, for example, any degree between 1° C. to 10° C., at 15° C., or more or less above or below internal body temperature for example, or ranges including any two of the foregoing values. It can also be desirable to deliver gases of a desired fixed or variable humidity and/or a desired fixed or variable temperature. The gases at the desired gas temperature and/or humidity can be dry cold gas, dry warm gas, humidified cold gas, or humidified warm gas for example.

Gases delivered into the patient's body can be relatively dry, which can cause damage to the body cavity, such as for example cell desiccation, cell death or adhesions. In some examples, a humidifier may optionally be operatively coupled to the insufflator. A controller of the system can energize a heater of the humidifier located in the gases flow path to deliver humidification fluid to the gases stream prior to entering the patient's body cavity. The humidification fluid may be water. The humidified gas can be delivered to the patient via further tubing which may also be heated. The insufflator and humidifier can be located in separate housings that are connected together via suitable tubing and/or electrical connections, or located in a common housing arranged to be connected to a remote fluid supply via suitable tubing.

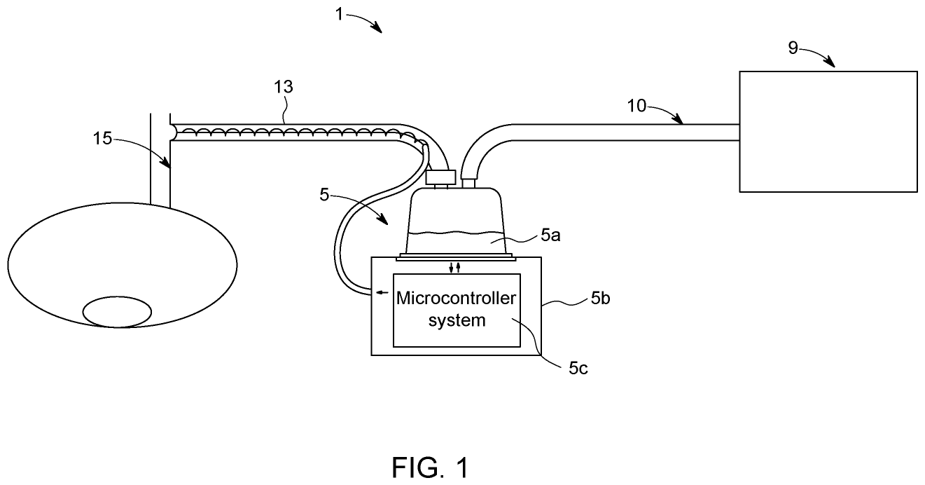

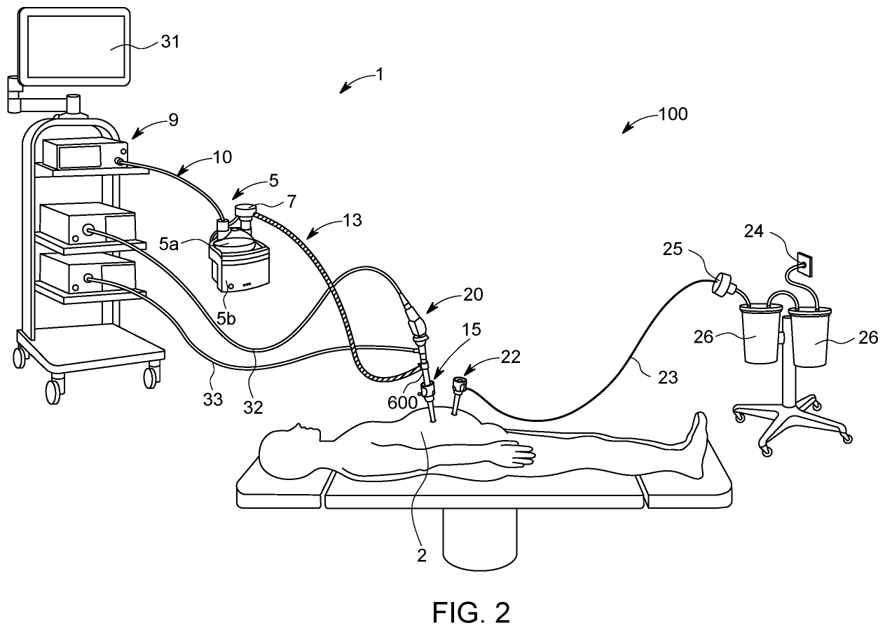

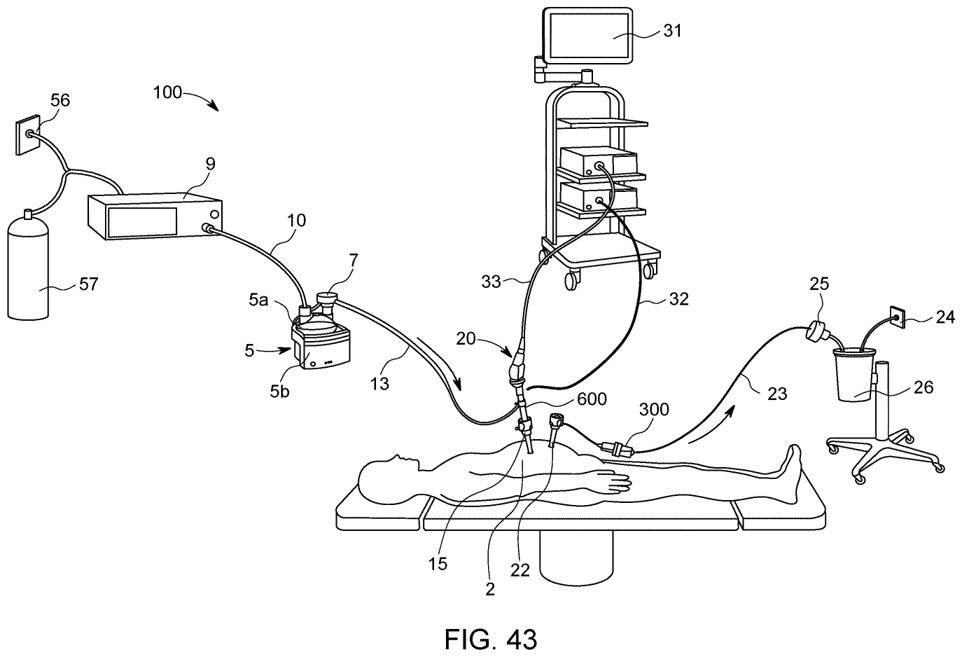

An example medical gases delivery system is shown in FIG. 1. An example surgical system 100, including the medical gases delivery system 1 during a medical procedure is shown in FIG. 2. A further example configuration for the surgical system 100, including additional system components, is shown in FIG. 43.

In some examples, the surgical system 100 can comprise a fluid source, such as a flow regulator 9 (for example, an insufflator), and a medical instrument 20 configured to be inserted into a body cavity 2, for example a surgical cavity within a patient, such as via a cannula 15. The flow regulator 9 may include or be associated with a flow generator, such as a blower. The flow regulator 9 may be associated with an appropriate supply of fluid, such as a gas bottle 57 or wall source 56, as shown in FIG. 43, for example. It will be appreciated that a fluid as referred to herein may refer to any gas or liquid or a combination thereof.

A humidifier 5 may be located between the fluid source (and the flow regulator 9) and the body cavity 2. Various styles or types of humidifiers may be used in combination with the other elements of the surgical system 100. In some examples, the humidifier may include a chamber (for example, a “pass-over” humidifier). However, other humidifier types are also contemplated, such as a humidifier including a wicking or other suitable absorbent material for holding humidification fluid. The wicking or other suitable absorbent material may be located in, for example, tubing or other equipment such as cannula.

The humidifier may be configured for use outside a sterile field of the operating room, such as shown in FIG. 2. The humidifier may be configured for use within the sterile field of the operating room. For example, the humidifier may be a patient-proximal humidifier, such as humidifier integrated into a cannula.

In some examples, a suitable humidifier may comprise a humidifier chamber 5a or medium to hold humidification fluid, and a heater configured to heat at least one of gas and the humidification fluid. In some examples, the humidifier 5 may comprise a heater base 5b and a chamber 5a. Examples of humidifiers are described in further detail in PCT/NZ 2015/050045, filed Apr. 16, 2015, and Application No. US Ser. No. 14/023,391, filed Sep. 10, 2013, the disclosures of which are hereby incorporated by reference in their entirety.

FIG. 4 shows one example of a humidifier chamber 5a, although other styles of humidifier chambers may be used. In general, suitable humidifier chambers comprise at least an inlet 5ab, an outlet 5aa and are configured to hold a volume of humidification fluid. The humidifier chamber may comprise a heater plate and/or heat conductive casing arranged to at least partially enclose the humidifier chamber. In the illustrated example, the humidifier chamber 5a and the gases inlet 5ab are configured to introduce the gases flow to the humidifier chamber at a direction substantially tangential or adjacent to the side wall of the humidifier chamber, such that the gases flow entering the humidifier chamber spins around the inside of the humidifier chamber or swirls within the humidifier chamber before exiting through the outlet 5aa. Such an effect can increase residence time of the gases within the humidifier chamber. In the example shown in FIG. 4, the gases inlet 5ab is orientated relative to a side wall of the humidifier chamber to introduce the gases flow to the humidifier chamber at a direction substantially tangential to the side wall of the humidifier chamber. The gases outlet 5aa is disposed in a top of the humidifier chamber, at or proximate to the centre thereof. The gases outlet 5aa may have an inner diameter greater than an inner diameter of the gases inlet 5ab. Humidifier chambers configured to spin or swirl gases flow entering the humidifier chamber before exiting through the outlet are described in further detail in PCT/NZ 2019/050032, filed Mar. 26, 2019 the disclosure of which is hereby incorporated by reference in its entirety.

The humidifier 5 may include a controller 5c configured to control one or more functions of the humidifier, including heating of a heating element, such as heater plate of the heater base 5b, for example. The controller 5c may be configured to include a warming function. The humidifier 5 may be configured such that connection of the tube 13 to the humidifier chamber outlet 5aa triggers warming of the heater plate. For example, the humidifier 5 may include one or more sensors communicatively connected with the controller 5c and configured to sense connection of the tube 13 to the humidifier chamber 5a.

The humidifier controller 5c may be configured to activate a warming mode of the humidifier 5 upon connection of the tube 13, to warm the heating element for a predetermined period of time. After the predetermined period of time has elapsed, the humidifier controller 5c may be configured to activate an operational mode of the humidifier 5. In the operational mode, the heating element of the humidifier 5 may be heated to a predetermined operational temperature.

The controller 5c may be configured to cause the humidifier 5 to enter a standby mode when the tube 13 is disconnected from the humidifier chamber 5a. The controller 5c may be configured to maintain the operational temperature of the heater plate of the humidifier 5 until the tube 13 is unplugged, or until the humidifier 5 is manually set to standby mode or switched off. Other mechanisms for warming and operation of the humidifier 5 are also possible.

The gases may be delivered via one or more delivery tubes 10, 13 in fluid connection with the humidifier. One or both tubes may heat or cool the gases as they travel between the flow regulator 9 and the body cavity 2. The tube 10 may deliver gases between the flow regulator 9 and the humidifier 5. The tube 13 may deliver humidified gases between the humidifier 5 and the body cavity 2.

In some examples, the tube 13 comprises a heated tube configured for connection to an outlet of the chamber 5a of the humidifier 5. The tube 13 may comprise a heating element provided within, throughout or around the tube. For example, the tube 13 may include a heating element or heater wire with a given resistance and/or a dedicated resistor. Tube resistance may be used to determine the current or power being put through the tube. A resistor within the tube may permit tube identification, for example. The humidifier 5 may be configured to detect connection of the heated tube 13 to the chamber outlet 5a.

Any of the fluid delivery tubes discussed herein may include a heating element. The system may be configured such that heating of each tube may be independently controlled and/or controlled in conjunction with one or more other system components such as the other tubes, the humidifier and the insufflator.

A heating element tube may be included in any tube where it is desired to maintain a temperature and/or humidity of the gas and/or to avoid condensation.

The tube(s) can additionally or alternatively include at least a portion comprising a breathable material which is permeable to water molecules, yet relatively impermeable to liquid water, respiratory gases and/or pathogens. Water molecules within the lumen of such tubes may be diffused through the breathable material. The water molecules can be desorbed to ambient air according to a gradient moving from the higher humidity side to the lower humidity side. This is known as the solution-diffusion mechanism (which can be distinguished from the pore-flow mechanism of porous membranes). Breathable tubes may incorporate a heating element or may be unheated. Examples of breathable tubes are disclosed in, for example, US6769431, US10532177 and PCT/NZ2023/050040.

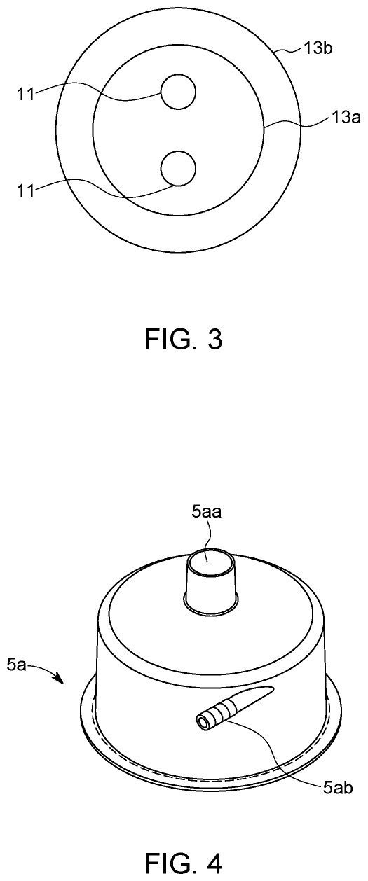

FIG. 3 shows one example of a configuration for delivery tube 13, although other tube configurations are also contemplated. The tube 10 may have any of the features described herein in relation to tube 13. The delivery tube 13 can be a flexible tube, made of a flexible plastic or other suitable material(s). Tube 13 may have a heating element e.g. heating wire, as explained above. FIG. 3 shows one configuration of a tube with a heating element in the form of heating wires.

Tube 13 may be provided in any suitable configuration. In the example shown in FIG. 3, tube 13 comprises inner and outer tubes 13a and 13b. Tube 13 may similarly comprise a wall having inner and outer wall 13a, 13b. The delivery tube 13 is configured to deliver gases through the lumen of the inner tube 13a. The tube 13 includes heater wires 11, positioned within the lumen of the inner tube 13a. The heater wires heat the gases as they travel between the gases source and the body cavity. A gap between inner tube 13a and outer tube 13b provides insulation to the inner tube 13a and the gases travelling therein. In some cases, the outer tube 13b may be corrugated. In some cases, the inner tube 13a may comprise a smooth bore.

The gases may pass through one or more filters, or filter units, in fluid connection with the tubes 10 and/or 13. For example, a filter may be provided downstream of the gases source/flow regulator. Alternatively or additionally, a filter 7 may be provided downstream of an outlet of the humidifier 5. Filter 7 may be in fluid connection with the outlet of the humidifier chamber 5a and the tube 13 to filter the humidified gas, for example as shown in FIG. 1. In some examples, the filter 7 may be associated with a heating element, which is operable to heat gases passing through the filter 7.

The system 1 may include one or more patient interfaces for introducing fluid to the body cavity 2, such as cannula 15 and/or medical instrument accessory 600. In some examples cannula 15 may be used to deliver gases into the body cavity 2.

The cannula 15 can include one or more passages to introduce gases and/or one or more medical instruments 20 into the body cavity 2. The medical instrument may be any appropriate instrument for use within the body cavity 2, such as a scope, an electro-surgery tool (such as an electrocautery tool), an energy, laser-cutting and/or cauterizing tool, grasping tools, or the like.

The system may have functionality for suction and/or venting of surgical smoke, or the like. This functionality may be provided via a venting path. The surgical system 100 may include one or more patient interfaces for venting fluid from the body cavity 2, such as one or more venting cannulas 22. Venting may be provided via evacuation line 23 in fluid connection with the venting cannula 22. The evacuation line 23 may be configured for connection to a suction source, such as a theatre wall suction port 24 or other suction source, such as mobile suction unit/vacuum generator, for example.

One or more suction canisters 26 may be provided in fluid connection with the evacuation line 23. In some examples, a single suction canister 26 may be provided for venting of gases and/or surgical smoke and for evacuation of irrigation fluid. The suction canister 26 may comprise a single inlet port. The use of a single canister for all evacuated gases and irrigation fluid may reduce cost and/or waste associated with performing the surgical procedure. In other examples, for example where evacuation of irrigation fluid is not required, the system may employ passive venting.

The evacuation line may be in connection with one or more filters, such as smoke filter 25. In some cases, venting may be provided via the same cannula providing insufflation gas to the body cavity 2.

The surgical system 100 can include monitoring equipment that is used together with the system. For example, a surgical scope (hereinafter referred to as a “scope”), such as a laparoscope may be used with, or be part of, the medical instrument 20 to allow displaying images recorded by the scope on an external monitor 31. The scope may be connected to the camera via camera feed line 32 and to a light source via light source line 33.

The surgical system 100 may include a medical instrument accessory 600. The medical instrument accessory may be configured to receive the medical instrument 20. The medical instrument accessory 600 may be used with the medical gases delivery or laparoscopic system, for example with cannula 15 of the surgical system 1 of FIG. 1. The medical instrument 20 and the medical instrument accessory may be introduced to the body cavity via the cannula 15. The medical instrument accessory may be configurable to direct gas flow relative to a medical instrument 20, e.g. a scope. This may reduce or prevent vision problems arising from condensation and/or surgical smoke. More detailed examples of medical instrument accessories are described below.

The surgical system 1 may include a flow controller as described in more detail herein (such as flow controller 300 or 3300) in connection with the evacuation line 23, for controlling flow in the evacuation line 23. The flow controller 300 may be positioned anywhere in the venting path, downstream of the body cavity 2. In the example of FIG. 43, the flow controller 300 is positioned close to the body cavity 2, relative to the length of the evacuation line 23. For example, a proximal portion of the evacuation line 23 between the flow controller 300 and a patient interface, such as venting cannula 22, may be shorter than a portion of the evacuation line 23 between the flow controller 300 and the suction source 24.

The flow controller 300 may be manually operable by surgical staff, e.g. a surgeon. The flow controller 300 may be positioned proximal to the body cavity 2, and inside the operating theatre sterile zone. This may enable easy access for a medical professional (such as a surgeon or surgical assistant, for example) to access the flow controller 300 to increase or decrease fluid flow in the evacuation line 23. More detailed examples of flow controllers 300, 3300 are discussed below.

In some instances, reference numerals of the same or substantially the same features may share the same last two digits.

### Examples of Medical Instrument Accessory

FIGS. 2 and 43 show one example of a medical instrument accessory 600 in use with cannula 15 of the surgical system 100. The medical instrument accessory 600 may be configurable to direct gas flow relative to a medical instrument 20, e.g. a scope. This may reduce or prevent vision problems arising from condensation and/or surgical smoke. The present disclosure provides further examples of medical instrument accessories 610a, 610b, 700, 800, 900, 1000, 1100, 1200, 1300, 1400, 1500, 1600, 1700, 1800, 1900, 2000, 2100, 2200 which may be suitable for use with the surgical system 100.

A surgical system, for example, including an insufflation system for supplying insufflation gases to a body cavity, for example as described above with reference to FIGS. 1, 2 and 43, can incorporate any of the example medical instrument accessories 600, 610a, 610b, 700, 800, 900, 1000, 1100, 1200, 1300, 1400, 1500, 1600, 1700, 1800, 1900, 2000 disclosed herein.

The example medical instrument accessories disclosed herein may be retro-fitted to existing surgical systems, for example, insufflation systems, without requiring bespoke customization (for example, bespoke connectors). The example medical instrument accessories disclosed herein can therefore enhance optical clarity of a scope viewing portion and/or maintain a clear field of vision during use. This may aid in minimizing operation duration and reduce number/frequency of interventions (such as withdrawal of the medical instrument to clean a viewing portion, for example). Further, the example medical instrument accessories disclosed herein can make it easier for the medical personnel, such as a surgeon, in operating a medical instrument during the medical procedure.

Delivery of gas flow close to, around or adjacent the distal end of the medical instrument, e.g. scope, may heat the end of the instrument. This may inhibit condensation forming by affecting the environment on and immediately around the scope viewing portion. The directed fluid flow may create a “micro-environment” around the viewing portion of the medical instrument that is at least partly isolated from the environment of the body cavity.

Additionally and/or alternatively, this may propel smoke particles away from the field of view of the medical instrument. This may be achieved by manipulating fluid flows, temperatures, and/or humidity in the micro-environment. This can advantageously maintain the temperature of scope viewing portion (or other instrument component, such as a sensor) above the dew point of the gas in the zone adjacent to the scope viewing portion.

The medical instrument accessory can be single use (disposable) or reusable. Alternatively, parts of the medical instrument accessory can be single use (disposable) or reusable. The medical instrument accessory may be made of materials that are biocompatible and/or sterilizable.

During laparoscopic surgery, there may be some form of electrosurgery/electrocautery/ultrasonic or laser device surgery to cause cutting or coagulation within the insufflated body cavity. This can produce surgical smoke within the cavity, and may become concentrated especially when there are no significant or sufficient gas leaks or suction/irrigation. A high concentration of smoke in the insufflated cavity, or a smoke plume moving towards a viewing portion of a scope in the cavity, can significantly impede optical clarity and field of vision for an operator of the scope, such as a surgeon or other member of a surgical team. In the absence of venting or suction, surgeons typically release all, or a portion of, the gas from inside the cavity, then re-insufflate. Surgeons may vent or use suction to extract the smoke and/or reduce the concentration of smoke by insufflating the cavity with clean gas.

Directing gas flow relative to a viewing portion of a scope can advantageously mitigate the effect of concentrated smoke in the insufflated cavity on visibility for a user operating the scope, for example, by affecting the environment immediately adjacent to the viewing portion. The directed gas may propel the smoke away from the viewing portion of the scope, to clear the line of sight and enhance the surgeon's field of vision. This may also prevent a smoke plume from contacting the medical instrument.

In some cases, a directed gas flow may facilitate directing gas closer to areas of interest. For example, a directed gas flow may reduce or eliminate stagnation zones of gas flow around the scope viewing portion. This may cause clean gas to be pushed into the field of vision and/or dilute the smoke with clean insufflation gas, both of which can improve optical clarity. The gas may be directed to specific areas of interest to assist in clearing the smoke in that area, which may then enable smoke to be more easily evacuated from the cavity.

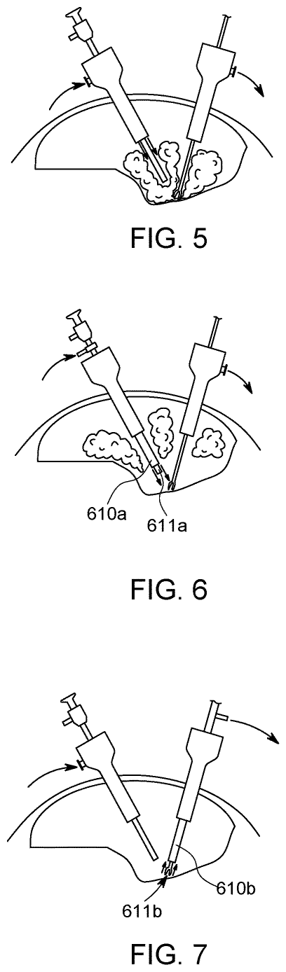

In some cases, venting adjacent the viewing portion of the scope can effectively remove smoke by venting gas from close to the smoke source. This can allow removing gas within or around the field of vision. Similarly, venting adjacent a distal portion of an instrument such as electrocautery device, may also assist to remove smoke by venting gas from close to the smoke source. FIGS. 5-7 illustrate conveying gases to, and venting gases from, adjacent the distal end of the medical instrument, e.g. a scope. This can advantageously allow affecting the environment immediately surrounding the viewing portion, regardless of depth of insertion of the medical instrument into the cavity and beyond the distal end of the cannula or accessory.

In some examples, the medical instrument accessory may comprise guide elements configured to position the medical instrument substantially concentrically relative to the accessory, which are described in further detail below. Furthermore, some examples are configured to direct gases relative to the viewing portion. Other examples may be configured to direct gases in front of the viewing portion and/or away from the viewing portion.

FIG. 5 illustrates a body cavity without use of a medical instrument accessory as described herein, such that gas/smoke is not being effectively cleared away. FIG. 6 illustrates the use of a first medical instrument accessory 610a to provide directed gas flow relative to the distal end of a medical instrument 611a which may include a scope or camera. FIG. 7 illustrates the use of a second medical instrument accessory 610b to provide venting relative to the distal end of a second medical instrument 611b. In some examples, two medical instrument accessories (e.g. 610a and 610b) may be used simultaneously to provide both directed gas flow and venting relative to the distal ends of two medical instruments (such as 611a and 611b). In other examples, a single medical instrument accessory may provide both directed gas flow and venting functions.

In FIGS. 6 and 7, a medical instrument is inserted into a cannula, which may be an insufflation cannula or a venting cannula. In other examples, a medical instrument may be inserted into a separate cannula (which may not be directed towards the pocket/s of smoke) from the insufflation and venting cannulas. The gas exiting from the insufflation cannula may be directed into the body cavity and may be relatively far from the pocket/s of smoke, which can result in stagnation. By contrast, a medical instrument accessory according to examples of the present disclosure may direct gas flow towards, vent gas from, and/or be positioned within the pocket/s of smoke.

### Examples of Seal and Rotatable Connection

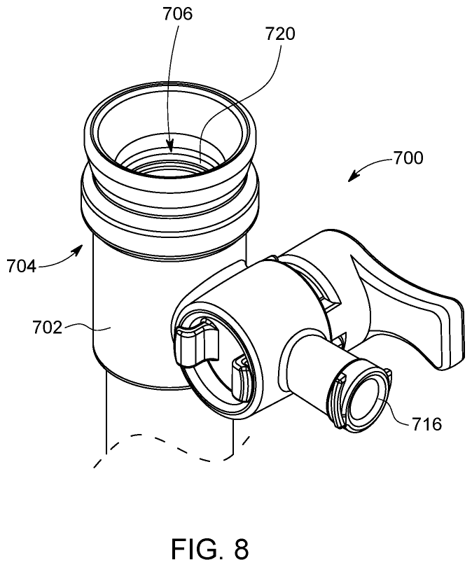

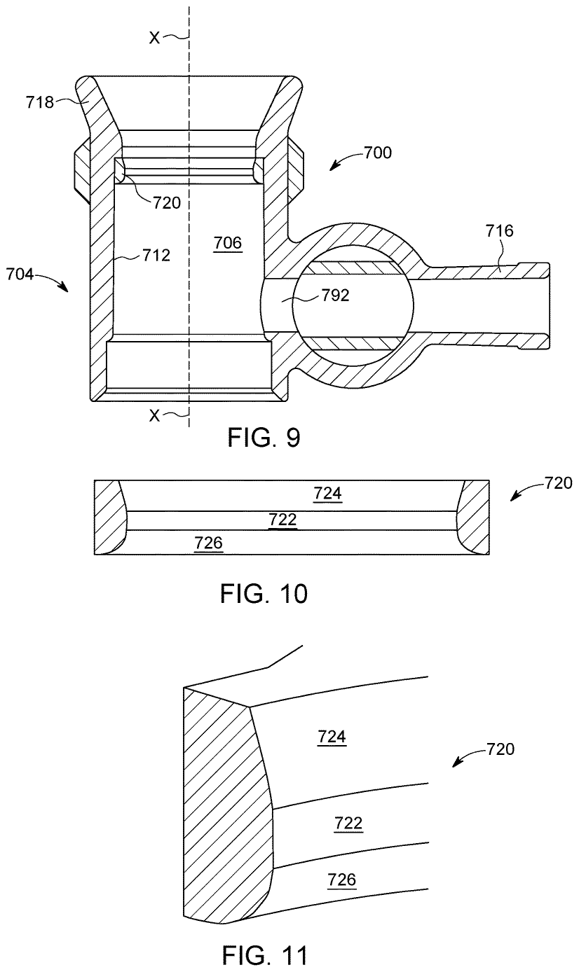

FIGS. 8-14 illustrate a medical instrument accessory 700 for directing fluid flow at, around or adjacent a distal end of a medical instrument. The accessory 700 can be positioned around a medical instrument and is securable to the medical instrument.

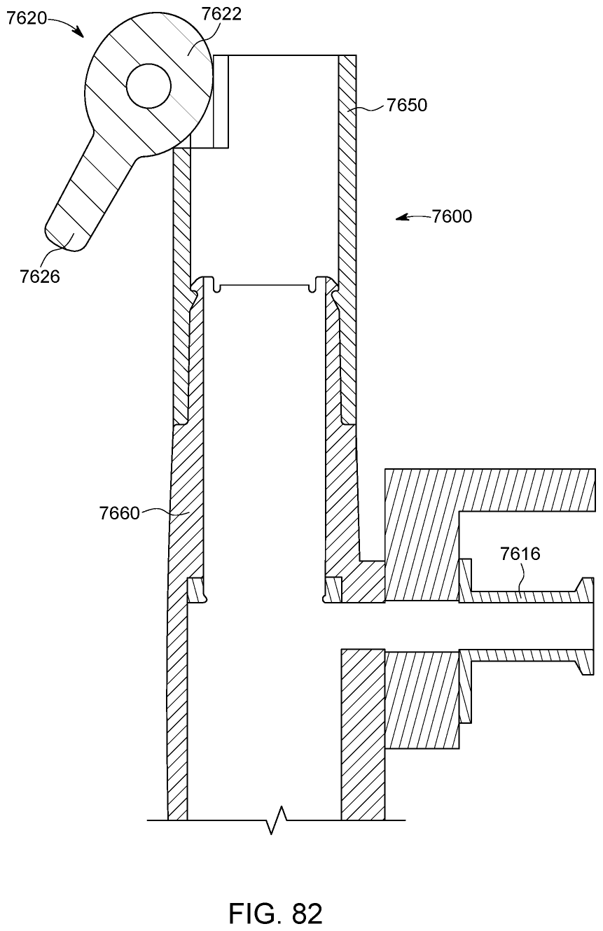

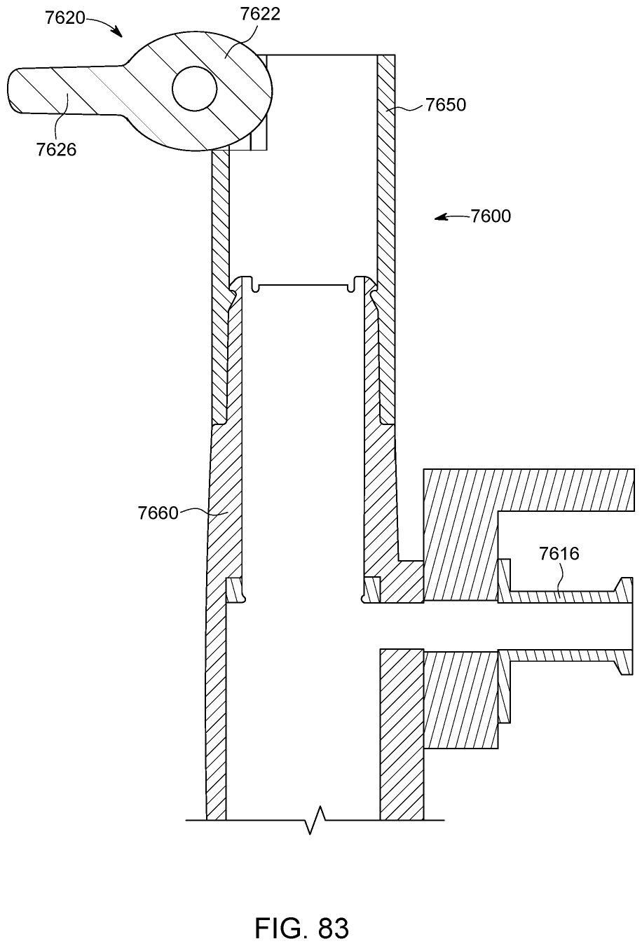

The accessory 700 may be securable to the medical instrument by a securing element. In some examples, a seal of the accessory 700 may function as the securing element. Additionally or alternatively, the accessory 700 may include a non-sealing securing element separate from a sealing element. For example, the accessory may include a locking mechanism, such as a cam-lock, as discussed in further detail in relation to FIGS. 82 and 83 below. The securing element may include a clamp, collar, or other similar structure, which may be positioned adjacent the proximal end opening of the medical instrument accessory and tightened to hold the accessory on the medical instrument.

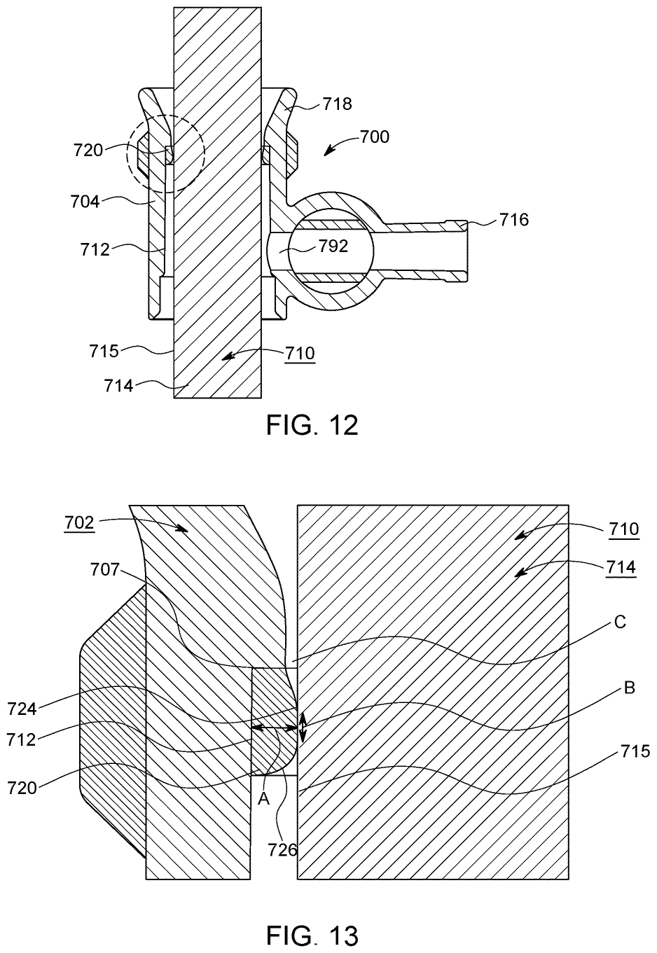

The medical instrument accessory 700 includes a body 704 mountable over at least a portion of a medical instrument 710, for example, over the shaft of a scope. At least part of the medical instrument accessory 700 may be movable (e.g. rotatable) relative to the medical instrument 710 during the surgical procedure. The body 704 includes an elongate shaft 702 extending from a proximal end to a distal end. The shaft 702 may define one or more lumens. In the example shown in FIGS. 12 and 13, the shaft 702 defines an inner lumen 706 dimensioned to at least partially receive a shaft 714 of the medical instrument 710. The one or more lumens can extend from, and be in fluid communication with, an opening or outlet defined in a distal end of the body 704. The lumen 706 is at least partially defined by an inner wall 712 of the body 704. When the medical instrument accessory 700 is mounted over the medical instrument shaft 714, a fluid flow path is defined between an outer wall 715 of the medical instrument shaft 714 and the inner wall 712 of the body 704.

In other examples, the fluid flow path may be defined by an additional lumen. For example, the inner lumen 706 may be configured to receive a medical instrument, without providing fluid flow while the additional lumen may provide the fluid flow path. In such examples, the additional lumen may be concentric with, or offset to, the inner lumen 706.

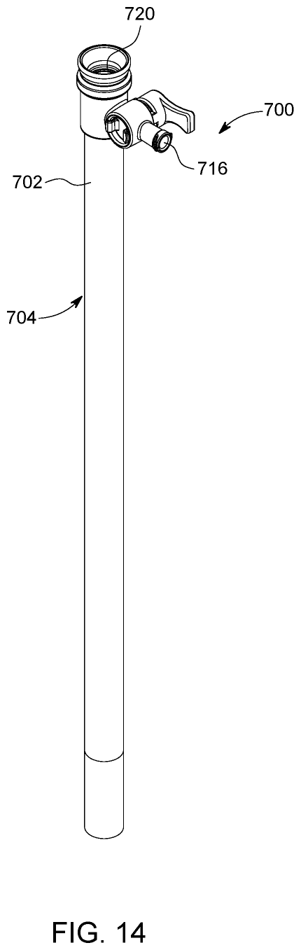

FIGS. 8, 9 and 12 show a proximal portion of the body 704. The body 704 may be dimensioned to extend further distally, or be connected to an additional distally extending portion of the body. FIG. 14 shows a full view of the body 704. The accessory 700 may be secured to release gas at or adjacent to the distal end of the medical instrument 710.

The accessory 700 can allow a directed gas flow to be delivered closer to an area of interest. For example, the accessory 700 can release the gas closer to a field of vision of the medical instrument 710. In some cases, the body 704 can have a length that extends towards, but stops short of, the distal end of the medical instrument such that the distal end of the medical instrument 710 may extend distally past the distal end of the medical instrument accessory 700. The distal end of the medical instrument 710 may protrude at least partway out the open distal end of the accessory 700. In some examples, the distal end of the medical instrument 710 may protrude a desired distance beyond the distal end of the accessory 700. Positioning the distal end of the medical instrument 710 adjacent to and beyond the distal end of the accessory 700 may facilitate smoke evacuation, particularly when the medical instrument 710 includes a cutting tool (such as an electrocautery tool), which creates smoke in use. In some examples the body 704 can have a length that extends to the distal end or beyond the distal end of the medical instrument 710. In such examples, a distal end of the body 704 may be positioned adjacent to, or extend beyond, a distal end of the medical instrument 710. For example, the distal end of the medical instrument 710 may be positioned inside the accessory 700 adjacent to the distal end. In some cases, the body 704 can be adjusted with respect to the medical instrument 710 such that the distal end of the body 704 extends beyond the distal end of the medical instrument 710.

The body 704 can include a fluid connecting portion, such as gas port 716, configured to connect the lumen in fluid connection with a fluid source and/or vent, to allow gases to enter and/or exit from the body 704. The port 716 can be connectable to a gases delivery tube, the gas delivery tube in turn fluidly connectable to a gas source. For example, the port 716 can be connectable to a medical gases delivery system, such as an insufflation system (such as any of the systems disclosed herein, for example). In the illustrated example, gas is delivered into the lumen 706 of the accessory 700 through a port, configured in this illustrated example as a gas inlet port 716. The accessory 700 can include one or more apertures 792 in the wall 712 of the body 704 that allow gas to pass from the inlet port 716 into the lumen 706. In other examples, the proximal end of the body 704 can be in fluid communication with the gas source.

The gas inlet port 716 may be opened and closed by movement of an actuator. In the illustrated example, the actuator comprises a spigot. The actuator may be rotated to open/close the port to provide gas flow to the lumen 706 of the accessory 700 as required.

FIG. 12 shows the medical instrument accessory 700 attached to the medical instrument shaft 714. In some examples, the body 704 may be configured to receive the medical instrument shaft 714 by sliding insertion of the medical instrument shaft 714 into the lumen 706 through the proximal end of the body 704. In some examples, the body 704 may include a guide portion 718 at its proximal end for receiving and guiding the medical instrument shaft 714 during insertion to the lumen 706. In the illustrated example, the guide portion 718 comprises a flared proximal portion of the body, which defines a funnel-shaped opening to the lumen 706.

The body 704 further comprises a sealing element 720. FIG. 8 shows an example sealing element 720 on inner surface of the shaft 702 of the body 704. In the example shown, sealing element 720 is over-moulded to the shaft 702. In some examples, sealing element 720 is not over-moulded to shaft 702. In some examples, the sealing element 720 is only positioned inwardly from the inner wall 712. That is, the sealing element 720 does not extend from the outside of the body 704.

The sealing element may perform a number of functions. The sealing element 720 is configured to provide a substantially fluid-tight seal between the medical instrument 710 and the medical instrument accessory 700. As well as sealing the fluid flow path, the sealing element 720 may be configured to provide resistance to movement of the medical instrument 710 relative to the accessory 700, for example, to retain the medical instrument 710 within the medical instrument accessory 700. As described in further detail below, the sealing element 720 may also allow for relative rotation between the sealing element 720 and the medical instrument 710, while maintaining the substantially fluid-tight seal. In some examples, the sealing element 720 may be configured to function as a securing element, to hold the accessory 700 in a desired position on the shaft of the medical instrument.

Additionally and/or alternatively the sealing element 720 may provide a higher resistance upon withdrawal of the medical instrument to the accessory than upon insertion of the medical instrument from the accessory 700. These various functions of the sealing element are discussed in further detail below.

As shown in FIG. 12, the sealing element provides a substantially fluid-tight seal between the inner wall 712 of the body 704 and the outer wall 715 of the medical instrument shaft 714. FIG. 13 shows an enlargement of a region of FIG. 12, indicated by the dashed circle. The sealing element 720 is configured to prevent gas from leaking out the proximal end of the lumen 706 such that the gas is released at the distal end of the accessory 700. The sealing element 720 may be positioned on the body 704 such that at least a portion of the sealing element 720 extends inwardly from the inner wall 712 of the body 704. The sealing element 720 may be positioned at or adjacent to a proximal end of the lumen 706 of the accessory 600. The sealing element 720 may be configured to attach at or adjacent to a proximal end of the medical instrument 710.

An example sealing element 720 is shown in more detail in FIGS. 10 and 11. The sealing element 720 comprises at least one contact surface configured to sealingly abut the outer wall 715 of the medical instrument shaft 714. In the example shown, the contact surface comprises an intermediate surface 722, located between a proximal surface 724 and a distal surface 726. In some examples, the contact surface may additionally comprise some or all of the proximal surface 724 and/or the distal surface 726. That is, some or all of the proximal surface 724 and/or the distal surface 726 may be configured to sealingly abut the outer wall 715 of the medical instrument shaft 714.

The sealing element 720 may be configured to maintain relative axial (longitudinal) positioning between the medical instrument 710 and the accessory 700. In such examples, a resistance to longitudinal movement of the sealing element 720 relative to the medical instrument shaft 714 may be high enough to withstand forces encountered during routine use of the medical instrument accessory 700 with the medical instrument 710, thus maintaining the relative axial positioning between the medical instrument 710 and the accessory 700 and retaining the medical instrument 710 within the accessory 700.

As mentioned above, the sealing element 720 may be configured to allow relative rotation between the sealing element 720 and the medical instrument shaft 714. This may allow for relative rotation between the inlet port 716 and the medical instrument 710. For example, the inlet port 716 may be rotatable about a longitudinal axis X of the body 704. In other examples, the inlet port 716 may be rotatable about an axis which is offset from the longitudinal axis X of the shaft.

The medical instrument accessory 700 may be configured to position the medical instrument 710 substantially concentrically within the lumen 706 of the medical instrument accessory 700, such that the inlet port 716 is rotatable around a longitudinal axis of the medical instrument shaft 714. In other examples, the medical instrument 710 may be offset (for example, linearly offset or at an angle) from the longitudinal axis X of the body 704.

In some examples, a resistance to rotational movement between the sealing element 720 and the medical instrument shaft 714 may be low enough to be overcome by forces encountered during routine use of the medical instrument accessory 700 with the medical instrument 710 (such as a torque produced by the weight of tubing attached to the inlet port 716, for example). This may allow the inlet port 716 to rotate substantially freely relative to the medical instrument 710. As such, movement of the inlet port 716 (and any attached tubes) may be substantially decoupled from movement of the medical instrument 710. This may enhance manoeuvrability of the medical instrument and attached medical instrument accessory, for example by reducing movement and drag of the attached tubes as a user manipulates the medical instrument 710.

The sealing element 720 may be configured to allow sliding insertion of the medical instrument shaft 714 to the lumen 706, through a central aperture of the sealing element 720. The medical instrument shaft 714 may be similarly removable by longitudinal sliding movement to withdraw the medical instrument 710 from the accessory 700. The sealing element 720 may be configured to provide resistance to the longitudinal movement of the medical instrument shaft 714 relative to the sealing element 720. The resistance to longitudinal movement of the sealing element 720 relative to the medical instrument shaft 714 may be low enough to be overcome by a user applying manual force to the medical instrument 710, such that the user may insert and/or withdraw the medical instrument 710 by hand.

Various configurations of the sealing element 720 may provide a first resistance to longitudinal movement during insertion of the medical instrument shaft 714 and a second resistance to longitudinal movement during withdrawal of the medical instrument shaft 714. The second resistance may be greater than the first resistance, such that it is easier to insert the medical instrument 710 than to withdraw it. Both the first and second resistance to longitudinal movement may be configured to be greater than the resistance to rotational movement of the seal 720 relative to the medical instrument shaft 714. In some cases, the first resistance may be greater than the second resistance and/or resistance to rotational movement of the seal 720 relative to the medical instrument shaft 714.

The sealing element 720 may be configured to provide a desired degree of resistance between the sealing element 720 and the outer wall 715 of the medical instrument shaft 714. A desired degree of resistance may be a resistance that is low enough and/or high enough to achieve the above-described functions of the sealing element 720, such as is described in further detail in relation to various examples below. As discussed in more detail below, one or more features or properties of the sealing element 720 may be configured to vary the resistance provided between the sealing element 720 and the medical instrument shaft 714.

A width, A, of a least a portion of the sealing element 720 (for example, a portion of the sealing element 720 positioned inwardly from the inner wall 712 of the body 704) may be adjusted to vary a degree of compression force between the sealing element contact surface and the outer wall of the medical instrument shaft when the sealing element 720 is engaged with the instrument shaft 714. For example, the resistance provided by the sealing element may be based at least in part on a width of the sealing element. The width of the sealing element is configured to be greater than a distance between the outer wall of the medical instrument shaft and the lumen inner wall, such that at least a portion of the sealing element is radially compressed between the outer wall of the medical instrument shaft and the lumen inner wall. Generally, a greater degree of compression will result in a higher resistance during insertion and/or withdrawal.

Further, a surface area, B, as shown in FIG. 13, of the contact surface (for example, intermediate surface 722) in contact with the outer wall 715 of the medical instrument shaft 714 may be adjusted to increase or decrease frictional resistance, by adjusting the size and/or geometry of the sealing element 720 to alter a cross-sectional profile shape of the sealing element 720.

Further still, material properties of the sealing element 720 may be configured to vary the resistance between the sealing element 710 and the medical instrument shaft 714. In some cases, surface treatments such as sandblasting or polishing may be applied to one or more contact surfaces (for example, at least the intermediate surface 722) to change (for example, reduce) frictional resistance. In other cases, one or more contact surfaces (for example, intermediate surface 722) may be roughened or otherwise treated to increase frictional resistance.

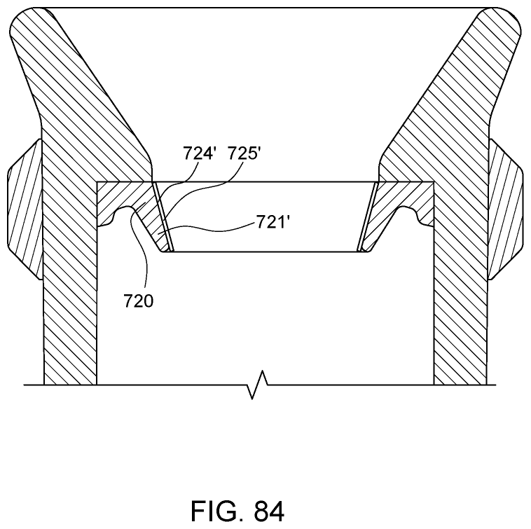

In the example shown in FIG. 84, sealing element 720′ includes a flexible portion 721′ configured to flex radially as the medical instrument shaft is inserted. The sealing element 720′ includes a proximal surface 724′ configured to contact an external surface of the medical instrument shaft. In this example, a friction reducing treatment 725′ (such as a coating, low-friction layer or other treatment configured to decrease frictional resistance) has been applied to the proximal surface 724′ to reduce friction between the sealing element 710′ and the medical instrument shaft upon insertion of the medical instrument. On withdrawal of the medical instrument shaft, the friction between the medical instrument shaft and the sealing element causes the sealing element 720′ to compress and/or fold on itself, increasing resistance to withdrawal. Additionally or alternatively, the sealing element may move during withdrawal such that the seal surface in contact with the shaft has increased frictional resistance compared to the proximal surface 724′.

In some cases, the sealing element 720 is configured such that movement of the medical instrument shaft in a proximal direction deforms the sealing element in a proximal direction. For example, the sealing element 720 may be at least partially formed from a material which is one or more of flexible, compressible, resilient and/or deformable. In some examples, the material may comprise a thermoplastic elastomer, although other suitable materials may also be used. Deformation of the sealing element 720 may increase the compression force and/or the surface area in contact with the surface 715 of the medical instrument 710, thus increasing resistance between the sealing element 720 and the medical instrument shaft 714.

In the illustrated example, the body 706 includes a radially inwardly extending abutment surface 707 abutting a proximal edge of the sealing element 720, which inhibits or limits movement of the sealing element 720 in the proximal direction. Upon withdrawal of the medical instrument shaft 714 from the lumen 706, the instrument shaft 714 will compress the sealing element 720 against the abutment surface 707.

In some examples, for example as shown in FIG. 13, the proximal surface 724 may be tapered, angled, curved or otherwise shaped to extend away from the surface 715 of the medical instrument shaft 714 when the accessory 700 is mounted over the medical instrument 710. The proximal surface 724 may be configured to at least partly define a proximal gap C between the proximal surface 724 and the outer surface 715 of the medical instrument 710.

Compression of the sealing element 720 against the abutment surface 707 may force the sealing element 720 partly into gap C. This may cause an increase in compression force and/or force of contact of the sealing element 720 against the shaft 714, due to more of the sealing element 720 material pushing against the shaft 714 and/or increase surface contact area of the sealing element 720 against the shaft 714, increasing the resistance between the sealing element 720 and the shaft 714.

The proximal surface 724 may define a funnel-shaped opening (in addition to the funnel-shaped opening defined by the flared guide portion of the body) configured to further guide the medical instrument shaft 714 into the lumen 706 during insertion. As such, a greater force is required to withdraw the medical instrument shaft 714 from the lumen 706 than to insert the medical instrument shaft into the lumen 706.

In some examples, the distal surface 726 of the sealing element 720 may be configured to define a distal gap between the medical instrument shaft surface 715 and the sealing element 720. The distal surface 726 may be configured to allow insertion and/or removal of the medical instrument shaft 714 with reduced friction and/or shearing on the sealing element 720. In some examples, the distal surface 726 may be tapered, angled, curved or otherwise shaped to extend away from the surface 715 of the medical instrument shaft 714 when the accessory 700 is mounted over the medical instrument 710.

In the illustrated example, the distal surface 726 comprises a convex fillet between the intermediate surface 722 and a distal end of the sealing element 720. The surfaces 722, 724 and/or 726 of the sealing element 720 may be substantially devoid of sharp corners, or transitions in curvature, which might catch on the instrument 710 during insertion and/or removal and tear the material of the sealing element 720. This may reduce or inhibit damage to the sealing element 720 during insertion and/or removal of the medical instrument 710.

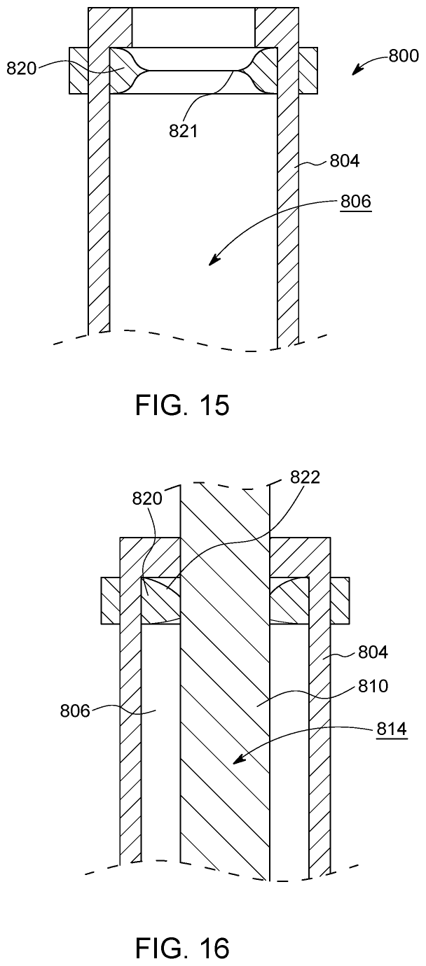

FIGS. 15 and 16 show an alternative seal profile. In this example, medical instrument accessory 800 includes a body 804 including a sealing element 820. The sealing element 820 is configured to seal against the shaft 814 of medical instrument 810. The sealing element 820 may include one or more of the features or characteristics as discussed above for sealing element 820. For example, as described with respect to sealing element 820, the sealing element 820 may be configured to allow relative rotation between the sealing element 820 and the medical instrument shaft 814, such that movement of the medical instrument 810 is decoupled from movement of a gas inlet (not shown) attached to the body 804. Further, the sealing element 820 may be configured to provide a resistance to longitudinal movement. The sealing element 820 may be configured to provide a first resistance to longitudinal movement during insertion of the medical instrument shaft 814 and a second resistance to longitudinal movement during withdrawal of the medical instrument shaft 814. In some examples, the first resistance may be lower than the second resistance. In some examples, the first resistance may be higher than the second resistance.

In contrast to sealing element 720, the sealing element 820 may include a tapered and/or pointed inner rim 821. The inner rim 821 flexes when the medical instrument shaft 814 is inserted to the lumen 806 to provide the sealing contact surface 822. Upon withdrawal of the medical instrument shaft 814 from the lumen 806, the instrument shaft 814 will deform the sealing element 820. This may cause an increase in compression force of the sealing element 820 against the shaft 814 thus making it harder to withdraw the instrument shaft 814 than to insert it. This can be due to a number of factors, such as an increase in surface contact area between the sealing element 820 against the instrument shaft 814 and/or a geometrical constraint of the sealing element 820 in at least one direction. The deformation of the sealing element 820 may result in increased radial compression and/or increased axial friction as a result of the change in the shape of the sealing element 820. In some examples, a surface of the sealing element 820 may be configured to provide increased friction upon withdrawal of the medical instrument shaft 814 compared to insertion. In some examples, a surface of the sealing element 820 may be configured to provide relative ease of deforming in one direction (e.g. insertion), and relative resistance to deformation, or return to a neutral position in a second direction (e.g. withdrawal).

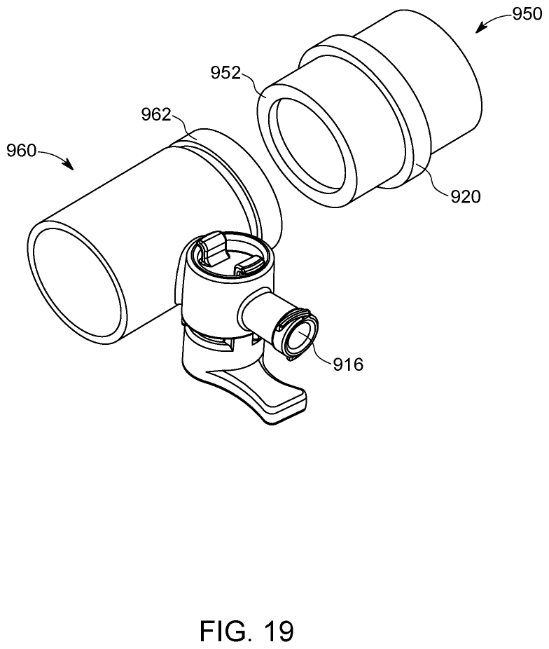

In some examples, a medical instrument accessory may include a body having a first portion and a least a second portion rotationally coupled to the first portion to allow for rotational movement between a fluid connecting portion of the medical instrument accessory and a medical instrument. In some examples, the body may include further rotatably coupled portions. For example, a sealing element may be comprised in the first portion, and the fluid connecting portion, such as an inlet port, may be comprised in the second portion. This may allow for rotational movement of the inlet port relative to the sealing element, such that the inlet port is rotationally decoupled from the sealing element. Examples of this are shown in the examples illustrated in FIGS. 15 to 22 and discussed in more detail below.

The first and second portions may comprise respective rotational coupling elements. The coupling elements may be configured to interlock such that the first and second portions are axially fixed relative to each other, but rotationally decoupled. That is, the coupling elements may inhibit or substantially prevent relative axial movement between the first and second portions but allow for relative rotation between the first and second portions. The interlocking of the coupling elements may also be configured to create a fluid-tight seal to inhibit fluids (for example, gases) from passing between the first and second portions and to prevent fluid from escaping at a proximal end of the accessory. For example, the coupling elements may be configured to mate in a substantially sealing manner, and/or be provided with a secondary seal element therebetween. The coupling elements may be configured to allow rotational movement between the first and second portions while maintaining the fluid-tight seal between the first and second portions and between the sealing element and the medical instrument shaft.

In such examples, the degree of frictional resistance between the sealing element and the medical instrument shaft may be configured to inhibit rotational movement between the sealing element and the medical instrument shaft, such that one portion (for example, the first portion) of the body is fixed to the medical instrument shaft by the seal. Alternatively, the sealing element may provide for a degree of rotational freedom relative to the medical instrument shaft in addition to the rotational freedom provided by the rotational coupling. In some cases, the rotatability of the medical instrument shaft relative to the sealing element may be determined by the friction force between the instrument and the seal, which may be configured by incorporating one or more of the features/properties of the sealing elements 620 or 720 discussed above.

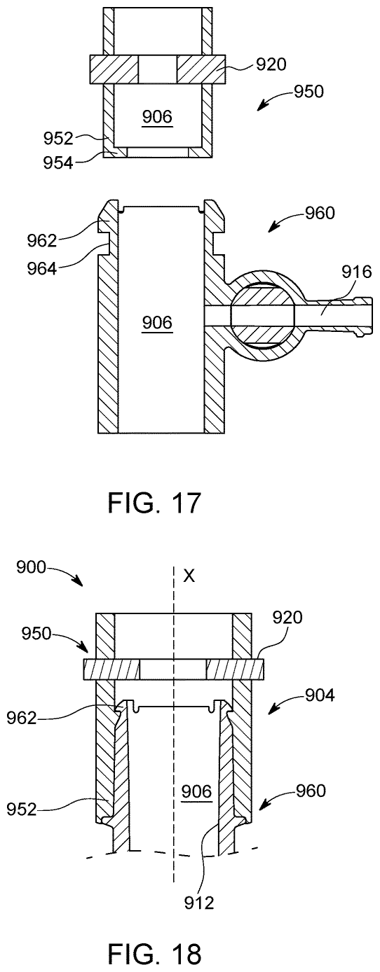

As one example, FIGS. 17-19 show a medical instrument accessory 900 for directing fluid flow around a distal end of a medical instrument. The medical instrument accessory 900 includes a body 904 mountable over at least a portion of a medical instrument (not shown), such as a scope. The body 904 extends from a proximal end to a distal end. The body 904 may define one or more lumens. In the illustrated example, the body 904 defines an inner lumen 906 dimensioned to at least partially receive a shaft of the medical instrument.

The one or more lumens can extend from, and be in fluid communication with, an opening or outlet defined in a distal end of the body 904. The lumen 906 is at least partially defined by an inner wall 912 of the body 904. When the medical instrument accessory 900 is mounted over the medical instrument shaft, a fluid flow path is defined between an outer wall of the medical instrument shaft and the inner wall 912.

FIGS. 17-19 show a proximal portion of the body 904. The body 904 includes a first portion 950, and a second portion 960, the second portion 960 rotatably coupled to the first portion 950. The first portion 950 and second portion 960 comprise a first conduit component and second conduit component respectively. The body 904 may be dimensioned to extend further distally, for example as indicated in FIG. 18, or may be connected to an additional distally extending portion of the body 904.

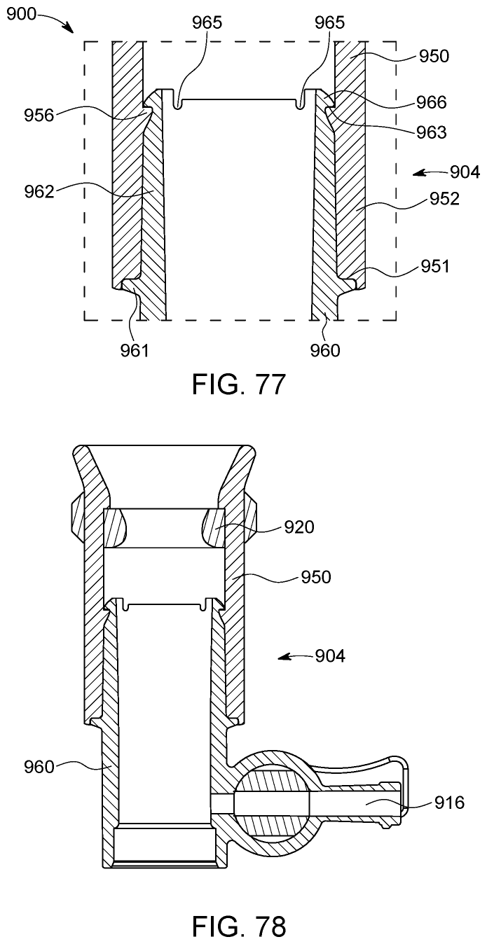

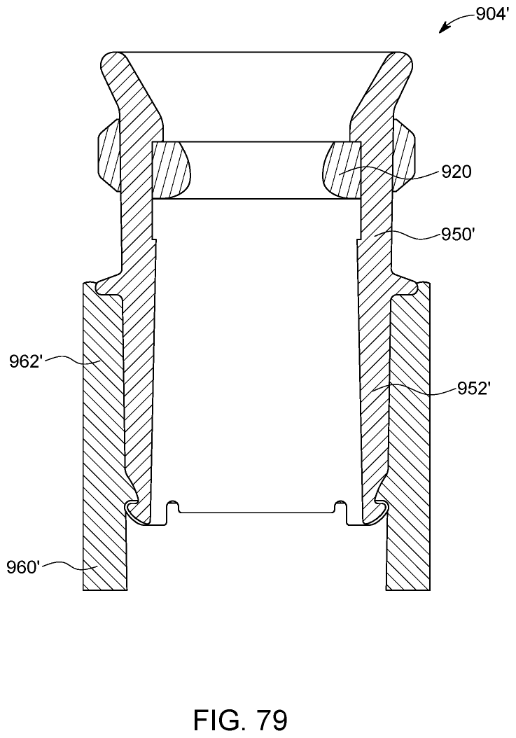

In the example shown in FIGS. 17-19, the first portion 950 and second portion 960 are configured to be connected together to form a swivel connection. The swivel connection is shown in further detail in FIGS. 77 and 78. The first portion 950 and second portion 960 are shown with at least part of the first portion 950 being arranged external to a proximal end of the second portion 960, and the proximal end of the second portion 960 being partially received in the first portion. That is, there is an internal connector component and an external connector component of the swivel connection. In the illustrated example, the first portion 950 includes a female (external) connector component 952 and the second portion 960 includes a male (internal) connector component 962. However, the reverse configuration is also contemplated. For example, in the body 904′ shown in FIG. 79, the first portion 950′ includes a male (internal) connector component 952′ and the second portion 960′ includes a female (external) connector component 962′.

The swivel connection allows relative rotation between the first portion 950 and second portion 960, and therefore allows relative rotation between the first portion 950 and gas inlet port 916.

The male and female connector components 952 and 962 include respective mating surfaces, configured to contact each other when the components are connected. One or more of the mating surfaces may be tapered.

The first portion 950, shown here as external component, comprises a wall with an interior surface. The second portion 960, shown here as internal component, comprises a wall with an exterior surface. The exterior surface may be substantially aligned with the interior surface. The exterior surface may be substantially parallel with the interior surface. One or both surfaces may be inclined relative to the other surface.

As can be seen in FIG. 77, the first portion 950 comprises an external shoulder or flange 951. The shoulder or flange 951 may be at or adjacent a distal end of the first portion 950. The shoulder or flange 951 may extend around a circumference of the exterior surface.

The second portion 960 comprises a shoulder or flange 961 on the exterior surface of the wall. When the first portion 950 and second portion 960 are assembled, the respective shoulders/flanges abut each other. The abutting connection of the shoulders/flanges functions as an end stop, limiting a distance that the second portion 960 can be inserted into the first portion 950.

The first portion 950 and second portion 960 may be connected together by a permanent interference fit, such as by a snap-fit connection.

The interior surface of the first portion 950 and exterior surface of the second portion 960 comprise engagement formations, which are engageable with each other to form a permanent connection. The connection features are configured such that the first portion 950 and second portion 960 can be assembled without requiring a significant amount of axial force (for example, enabling manual assembly), but cannot subsequently be readily disconnected. After assembly, the first and second portion 950, 960 are able to rotate independently from, and relative to, each other whilst maintaining a secure connection. The connection may be configured to minimise or avoid gas leak through the connection.

With continued reference to FIG. 77, the exterior surface of the second portion 960 has an engagement formation in the form of a protrusion 966 extending peripherally around its distal end. The second portion 960 tapers towards the distal end to define an undercut 963 extending radially inwardly, adjacent the protrusion 966. In other words, the protrusion 966 extends over the undercut 963. The protrusion 966 may be a radial protrusion.

The engagement formation with protrusion may form flexing portions or flexing arms. One or more slots may be provided to allow for easier flexure of the flexing arms, particularly as second portion 960 is inserted into the first portion 950 for connecting engagement. This may facilitate easier assembly of the first and second portions 950, 960. As shown in FIG. 77, the second portion 960 may comprise slots 965.

When viewed in cross-section, the protrusion 966 of the second portion 960 has a wedge shape with a sloped distal surface acting as a lead-in to facilitate insertion of the second portion 960 into the first portion 950. The protrusion 966 has a proximal surface that engages with the first portion 950. The proximal surface of the protrusion 966 (adjacent undercut 963) may have an angle (with reference to the axial or longitudinal direction), that contributes to the interference fit between the second portion 960 and first portion 950 and facilitates the permanent connection.

A ledge 956 is disposed on the interior surface of the first portion 950. The ledge may engage with the protrusion 966 on the distal end of the second portion 960. The protrusion 966 of the second portion 960 may have a greater external diameter than the ledge 952 of the first portion 950 such that there is an interference fit between the two components when connected, providing a permanent connection.

The proximal surface of the protrusion 966 of the second portion 960 abuts the ledge 956 of the first portion 950 when the two components are assembled, such that the first and second portions 950, 960 are rotatably connected. The ledge 956 may provide a bearing surface for the protrusion 966 wherein the proximal surface of the protrusion 966 can contact the ledge 956 in use. The contact between ledge 956 and protrusion 966 may also assist to minimise leakage of gases.

The accessory 900 can be dimensioned to extend to, or beyond, the distal end of the medical instrument and can be secured to release gas at the end of the medical instrument.

The accessory 900 can allow a directed gas flow to be delivered closer to an area of interest. For example, the accessory 900 can direct the gas closer to the field of vision of the medical instrument 910. In some cases, the body 904 can have a length that extends to the distal end or beyond the distal end of the medical instrument 910 such that the distal end of the body 904 is adjacent to or extends beyond a distal end of the medical instrument 910. The accessory 900 is configured to receive the medical instrument shaft, for example by sliding insertion of the medical instrument shaft into the lumen 906 through the proximal end of the body 904.

The first portion 950 includes a sealing element 920 configured to sealably secure the accessory 900 to the medical instrument. The sealing element 920 may be configured to attach to the outer wall of the medical instrument shaft. For example, the sealing element 920 may be configured to attach at or adjacent to a proximal end of the medical instrument. The sealing element 920 is configured to create a fluid-tight seal between the inner wall 912 of the body 904 and the outer wall of the medical instrument shaft. The sealing element 920 is configured to extend between the outer wall 914 of the medical instrument shaft and the lumen inner wall 912 to seal the fluid flow path. The sealing element 920 is provided to prevent gas from leaking out the proximal end of the lumen 906 such that the gas is released at the distal end of the accessory 900.

The sealing element 920 may be further configured to reduce, limit or inhibit relative movement (rotational and/or axial) between the medical instrument and the first portion 950. The sealing element may be rotationally and/or axially fixed relative to the first portion 950. It will be appreciated that, in some cases, the frictional resistance may be able to be overcome in order to slidingly insert and/or remove the medical instrument from the accessory 900.

However, the frictional resistance may be configured such that, under forces applied during normal use of the medical instrument, the first portion 950 may be effectively fixed to the medical instrument shaft.

Additionally or alternatively, the medical instrument accessory may include a discrete securing element fixed relative to a first portion of the medical instrument accessory body, which may be separate from a sealing element. FIGS. 82 and 83 illustrate a portion of an example medical instrument accessory 7600 including a securing element in the form of a locking mechanism 7620 arranged adjacent an opening defined at a proximal end of a first portion 7650 of the medical instrument accessory 7600.

In the example shown, the locking mechanism 7620 includes a cam 7622 which is rotatable about an axis to move between a first (open) position (shown in FIG. 82) and second (locking) position (shown in FIG. 83). The locking mechanism 7620 can be operated, for example, by a user operating a lever 7626 extending from the cam 7622. As shown in FIG. 82, the cam 7622 is shown in the open position so that the cam 7622 does not obstruct the opening and, consequently, does not interact with a medical instrument arranged within the accessory 7600. FIG. 83 illustrates the cam 7622 in the locked position to extend into the opening. Arranging the cam 7622 in the locked position allows the cam 7622 to interfere with the medical instrument to secure the instrument in the accessory 7600.

The first portion 7650 is rotatably connected to a second portion 7660 via a swivel connection, such as described in relation to FIG. 77, such that a gas inlet port 7616 of the medical instrument accessory 7600 is rotationally decoupled from the locking mechanism 7620.

The second portion 960 may comprise a fluid connecting portion, such as gas inlet port 916, configured to connect the lumen in fluid connection with a fluid source and/or vent, to allow gases to enter and/or exit from the body 904. The inlet port 916 can be connectable to a gases delivery tube of a gas source, for example, an insufflation system (such as any of the systems disclosed herein, for example). In the illustrated example, gas is delivered into the lumen 906 of the accessory 900 through a port, configured in this illustrated example as a gas inlet port 916. The accessory 900 can include one or more apertures (not shown) in the wall 912 of the body 904 that allow gas to pass from the inlet port into the lumen 906. In other examples, the proximal end of the body 904 can be in fluid communication with the gas source.

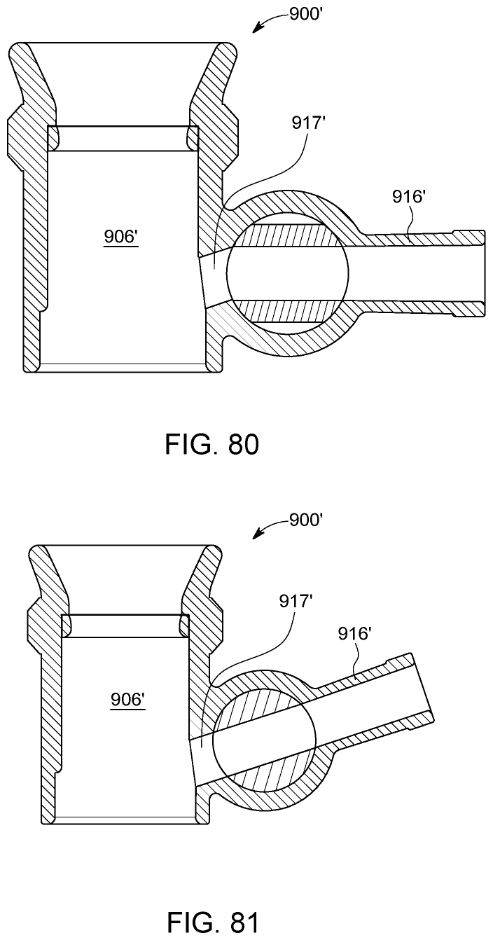

The gas inlet port 916 may be configured to be substantially perpendicular to the lumen 906 of the medical instrument accessory 900, as shown in FIG. 17 for example. In other examples, such as shown in FIGS. 80 and 81, the gas inlet port 916′, and/or an internal lumen 917′ of the gas inlet port, may be provided at an angle relative to the lumen 906′of the medical instrument accessory 900′. Providing the gas inlet port 916′ (and/or a lumen 917′ thereof) at an angle may facilitate delivery of fluid into the medical instrument accessory 900′ at an angle that is closer to the direction of intended flow than would be achieved with a perpendicular inlet. This may contribute to minimising leakage of fluid past the sealing element 920 by directing the flow away from the sealing element 910 and toward the distal opening of the medical instrument accessory 900′.

The gas inlet port 916 may fluidly connected to the lumen 906′ via a movable coupling, such as a ball joint coupling. Such a coupling may allow further variation of inlet angle and/or may provide some additional decoupling of gas inlet rotation relative to the medical instrument.

The rotatable coupling between the first portion 950 and the second portion 960 allows for relative rotational movement between the inlet port 916 and the medical instrument shaft 914. For example, the inlet port 916 may be rotatable about, and substantially rotatably decoupled from, the medical instrument shaft 914. As shown in FIG. 18, the first portion 950 and the second portion 960 are aligned with respect to each other along a longitudinal axis X. The first portion 950 is configured to secure the medical instrument shaft within the lumen 906. Since the second portion 960 comprises the inlet port 916, the inlet port 916 is configured to rotate freely relative to the medical instrument shaft 914 and the first portion 950. When mounted over the medical instrument, the lumen 906 may be concentric with an outer wall of the medical instrument shaft 914, such that the inlet port 916 is rotatable around a longitudinal axis of the medical instrument shaft 914 which is coincidental with the longitudinal axis X of the lumen 906. In other examples, a longitudinal axis of the medical instrument shaft may be offset from the longitudinal axis X of the lumen.

The first portion 950 and second portion 960 may comprise mating surfaces for guiding relative rotation and/or providing a fluid-tight seal. In the example illustrated in FIGS. 17-19, the first portion 950 comprises a female swivel connector portion 952 and the second portion 960 comprises a male swivel connector portion 962 (however, the reverse configuration of male and female connector portions is also contemplated). In this example, the female swivel connector portion 952 includes a projection 954, receivable in a groove 964 on the male connector portion 962. The groove 964 may be on an outer surface of the male connector portion 962. In some examples, the projection 954 and groove 964 may be circumferential. The male and female swivel connector portions 952 and 962 may be configured to interlock via a snap-fit connection. In other examples, the first portion 950 and second portion 960 may comprise retention features (for example, clips) to connect the first and second portions 950, 960.

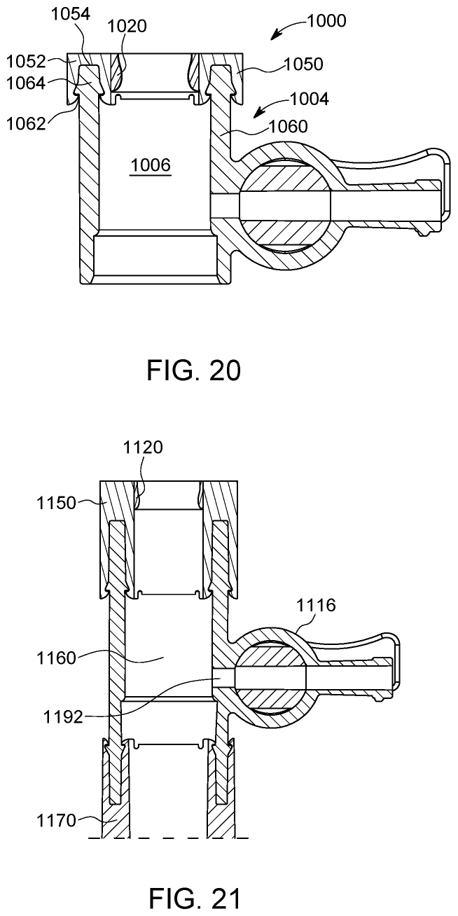

Further examples of a rotatable coupling between first and second portions of a body of a medical instrument accessory are illustrated in FIGS. 20-22.

FIG. 20 shows a cross-sectional view of a further arrangement for a swivel connection. A body 1004 of a medical instrument accessory 1000 may comprise a first portion 1050 and a second portion 1060. The first portion 1050 may be a first conduit component.

The second portion 1060 may be a second conduit component. In the example shown in FIG. 20, the swivel connection between first and second swivel connector portions 1050, 1060 is around, or substantially in the same plane as sealing element 1020. The first portion 1050 may comprise a female swivel connector portion 1052 and the second portion 1060 may comprise a male swivel connector portion 1062 (although the reverse configuration of male and female connector portions is also contemplated in this and other examples described herein).

The female swivel connector portion 1052 defines a groove 1054 configured to receive a correspondingly shaped projection 1064 on a proximal end of the male swivel connector portion 1062. The projection 1064 projects inwardly and outwardly with respect to the lumen 1006. The projection 1064 defines a pair of outwardly extending shoulders or flanges, defining an undercut on either side of the projection 1064. The undercuts are configured to abut a distal ledge of the groove 1054, when connected, to inhibit separation of the projection 1064 from the groove 1054. The ledge may provide a bearing surface for the protrusion 1064. The projection 1064 includes a sloped proximal surface, which facilitates insertion of the projection 1064 into the groove 1054.

In use, the first portion 1050 is fixed relative to a shaft of a medical instrument (not shown) by sealing element 1020 (for example, as described above with respect to sealing element 920 of FIGS. 17-19). The second portion 1060 comprises a gas inlet port, which may be substantially as described in relation to gas inlet port 916 in FIGS. 17-19. The rotatable coupling of the swivel connector portions 1052 and 1062 allows for rotation of the second portion 1060 relative to the first portion 1050 and the medical instrument (not shown).

FIG. 21 illustrates another arrangement for a swivel connection. A body 1104 of a medical instrument accessory 1100 may comprise a first portion 1150 and a second portion 1160, the second portion rotatably coupled to the first portion 1150. The second portion 1160 comprises gas inlet port 1192. In this example, the body 1104 may comprise a third portion 1170, distal to the first and second portions 1150, 1160.

The first portion 1150, the second portion 1160, and the third portion 1170 may rotate independently of each other. For example, the third portion 1170 may be rotatably connected to the second portion 1160 by a rotatable coupling such that the second portion 1160 may rotate independently of both the first portion 1150 and the third portion 1170. As shown in FIG. 21, the first portion 1150 may comprise a female swivel connector portion 1152 and the second portion 1160 may comprise a proximal male swivel connector portion 1162a. These swivel connector portions 1152, 1162a may interlock in a similar manner to swivel connector portions 1052 and 1062, as described in relation to FIG. 20 to allow rotational movement between the first and second portions 1150, 1160. The second portion 1160 further comprises a distal male swivel connector portion 1162b, which is configured to interlock with a female swivel connector portion 1172 of the third portion 1170. Gas inlet port 11192 may therefore be rotatable relative to first portion 1150 and/or third portion 1170.

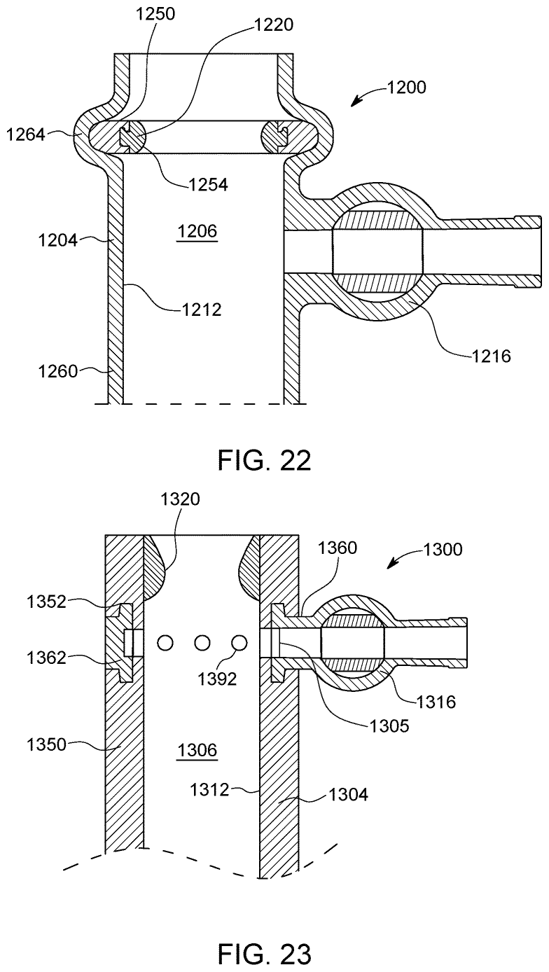

In the example illustrated in FIG. 22, the medical instrument accessory 1200 includes a body 1204 comprising a first portion 1250 comprising a circumferential ring 1254. A second portion 1260 comprises a circumferential recess 1264 in wall 1212, configured to receive the ring 1254. The first portion 1250 comprises a sealing element 1220, which is configured to seal against the shaft of a medical instrument (not shown). The sealing element 1220 may be further configured to fix the first portion 1250 relative to the medical instrument. In some examples, the sealing element 1220 may be integrally formed with the first portion 1250. For example, the first portion 1250 may include a semi-hard seal surface (such as a rubber material, for example), with lubrication in the recess 1264.

Continuing with reference to FIG. 22, the second portion 1260 comprises a gas inlet 1216.The rotatable coupling of the ring 1254 and recess 1264 allows for rotation of the second portion 1260, and thus the gas inlet 1216, relative to the first portion 1250 and the medical instrument. The second portion 1260 may extend from a proximal end towards a distal end of the medical instrument accessory 1200, defining both the proximal and distal portions of the lumen 1206. The second portion 1260 defines an external portion of the body 1204, while the first portion 1250 is located internally to the second portion 1260. As such, the entire outer length (as defined by the second portion 1260) of the body 1204 may rotate, together with the gas inlet 1216, relative to the medical instrument.

In the example shown in FIG. 23 a body 1304 of a medical instrument accessory 1300 may comprise a first portion 1350 and a second portion 1360. The first portion 1350 comprises a shaft 1312 defining a circumferential recess 1352 in an outer surface thereof. A second portion 1350 comprises a circumferential ring 1362, configured to be rotatably received in the recess 1352. In use, the first portion 1350 is fixed relative to a shaft of a medical instrument (not shown) by sealing element 1320. The second portion 1360 comprises a gas inlet 1316. The rotatable coupling of the ring 1362 and recess 1354 allows for rotation of the second portion 1360, and thus the gas inlet 1316, about the shaft 1312 of the first portion 1350.

The shaft 1312 defines a plurality of apertures 1392 extending from the recess 1354 through to a lumen 1306 defined by an inner wall 1312 of the body 1304. As shown in FIG. 23, the apertures 1392 may be in an annular array, spaced circumferentially about the lumen 1306. The apertures 1392 are aligned longitudinally with the gas inlet 1316 to allow gas to pass from the gas inlet 1316 into the lumen 1306 as the gas inlet rotates about the shaft 1312. The body 1304 may include a channel 1305 in fluid connection with the apertures 1392 and the gas inlet 1316 such that gas can flow into the lumen 1306 regardless of the rotational position of the first portion 1350 relative to the second portion 1360.

### Examples of Directed Gas Flow Around a Medical Instrument With a Medical Instrument Accessory

As previously discussed, it can be desirable to create a micro-environment at, around, near or adjacent a distal end of the medical instrument to overcome some of the condensation, fogging, or other issues that can cause reduced visibility. A directed gas flow around the medical instrument can allow for the creation of the micro-environment to be controlled around the viewing portion or working end of the medical instrument. This micro-environment can isolate the viewing portion from the warm and humid environment of the body cavity, e.g. pneumo-peritoneum. The medical instrument with the viewing portion can be either held concentrically or off-axis surrounded by a gas pathway. This may cause the insufflation gas to conform to, and may substantially enclose, the medical instrument. The gas may then cover the viewing portion of the medical instrument and, to a certain extent, form a barrier between the viewing portion and the surrounding environment. If the delivered gas conditions are controlled, this can allow affecting the environment around the medical instrument. Further, the gas flow can advantageously be directed to an area of interest by adjusting the positioning of the medical instrument accessory and/or medical instrument. For example, the gas flow can be directed to region(s) of the body cavity where there is smoke, including where smoke might otherwise settle or become stagnant.

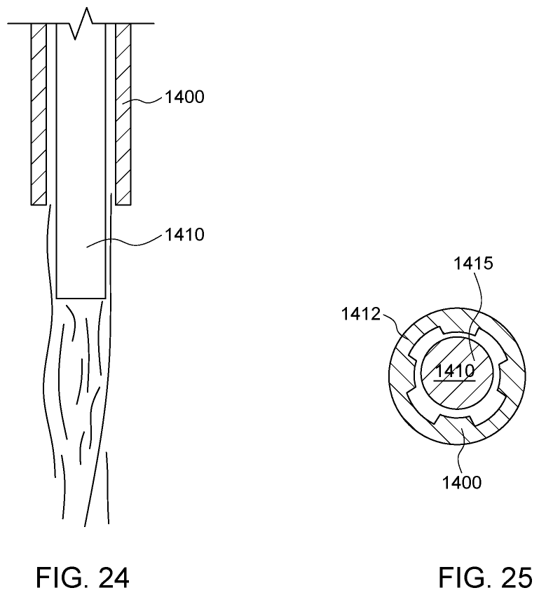

FIG. 24 illustrates directed gas flow around a medical instrument 1410 within a medical instrument accessory 1400. As shown in FIG. 25, the gas flows through one or more fluid flow paths defined between an outer surface 1415 of the medical instrument 1410 and an inner wall 1412 of the medical instrument accessory 1400.

In some cases, a medical instrument accessory can include at least one guide element to position the medical instrument relative to the lumen, such that a gas flow path is defined between the lumen inner wall and the medical instrument shaft. The medical instrument may be positioned concentric to a longitudinal axis defined by the lumen, or offset from the axis (for example, linearly offset or at an angle to the axis). The medical instrument accessory may include guide elements in combination with or addition to any of the above described features, for example, features relating to the sealing element or rotatable components.

Detailed examples of the guide elements that may also be used in combination or in addition to those described herein are described in International Application No. PCT/NZ2019/050100, titled “DIRECTED GAS FLOW SURGICAL CANNULA FOR PROVIDING GASES TO A PATIENT”, filed on Aug. 16, 2019, or in PCT/IB2021/05115, titled “DIRECTED GAS FLOW ACCESSORY FOR PROVIDING GASES TO AND VENTING GASES FROM A PATIENT”, filed on Dec. 2, 2021, the disclosures of which are hereby incorporated by reference in their entirety.

The at least one guide element may be on or within an inner wall of the accessory and configured to position the shaft of the medical instrument within the lumen. In some cases, the guide element(s) can be arranged at the inner wall of the accessory, such as being defined by the inner wall, or being mountable to or adjacent the inner wall. For example, the guide element(s) can extend inwardly from an inner wall of the shaft of the accessory. For example, guide element(s) may be separate from, and securable to, a shaft of the accessory to be arranged at the inner wall. The guide element(s) can extend inwardly relative to the inner wall such that, in use, the guide element(s) are positioned between the inner wall and the medical instrument. In some cases, the medical instrument accessory can have a plurality of the guide elements.

In some examples, the guide elements may extend partway along the longitudinal length of the inner wall of the accessory. It will be appreciated that the guide elements may be arranged adjacent one, or both, of the proximal and distal ends of the accessory, continuously between the ends, or discontinuously at spaced intervals between the ends. In some examples, the guide elements extend to the distal end of the accessory and terminate flush with the distal end of the inner wall. In other examples, a terminal end of one or more of the guide elements may be offset from the distal end of the accessory.

The guide elements, together with the inner wall and an outer surface of the medical instrument, may define one or more fluid flow paths, for example as shown in FIG. 24. The proximal end of the body of the medical instrument accessory can be in fluid communication with a gas source or vent. Gas can be directed out from the open distal end of the accessory and around an end of the medical instrument.

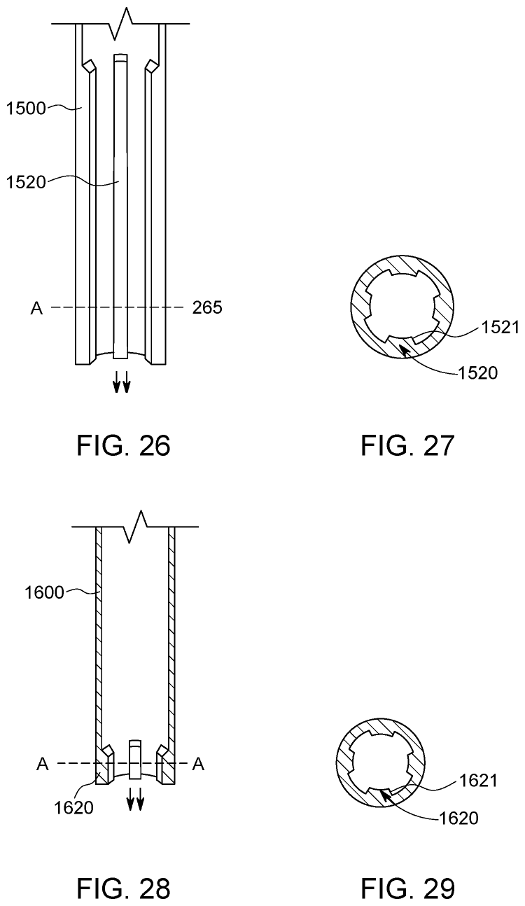

In some examples, the at least one guide element is formed by one or more ribs. As one example, FIG. 26 illustrates longitudinal (axially) extending ribs 1520 defined by an inner wall of a medical instrument accessory 1500. The ribs 1520 are arranged to substantially concentrically position the accessory 1500 around a medical instrument (not shown). FIG. 27 illustrates a cross-section through line A-A of FIG. 26 of the medical instrument accessory 1500. As shown in FIG. 27, the ribs 1520 can form an annular array spaced circumferentially around the inner wall to hold a medical instrument substantially concentrically in the medical instrument accessory 1500. The ribs 1520 are arranged such that gases can flow through the accessory 1500, between a sidewall 1521 of the ribs 1520, and around the outer surface of the medical instrument (not shown). In some cases, the ribs 1520 can extend inwardly from an inner wall of the medical instrument accessory 1500 as shown in FIG. 27. It will further be appreciated that configuring the guide elements as ribs 1520 is exemplary and these guide elements may be configured in other forms, such as one or more bumps, dimples, fins, splines, pins, grooves, or channels.

As illustrated in FIG. 28, ribs 1620 can be positioned at, or adjacent, an open distal end of a medical instrument accessory 1600. The ribs 1620 extend longitudinally, but to a lesser extent than the ribs 1520 of FIG. 26. The ribs 1620 can define a gas pathway for gas to travel around the periphery or circumference of the medical instrument. The ribs 1620 can hold the medical instrument substantially concentrically within the lumen of the medical instrument accessory. In some cases, one or more of the ribs may not contact the medical instrument while in use, but can act as limits or stops, to prevent the medical instrument from contacting the interior sidewall of the medical instrument accessory shaft. This may enable the gas pathway to remain open. FIG. 29 illustrates a cross-section through line A-A of FIG. 28 of the medical instrument accessory 1600. As shown in FIG. 29, the ribs can be spread radially to promote centering of the medical instrument in the medical instrument accessory 1600 and the gas flow can pass between the sidewall 1621 of the ribs 1620 around the medical instrument and the outer surface of the medical instrument (not shown). In some cases, the ribs 1620 can be spaced axially and/or longitudinally apart regularly or irregularly along the inner circumference of the medical instrument accessory 1600.

In some cases, the at least one guide element can include protrusions such as bumps or indents in the medical instrument accessory to direct gas flow concentrically around the medical instrument. The protrusions can extend inwardly from the inner wall of the lumen of the medical instrument. The protrusions can be located at any position along the body of the medical instrument accessory to position the medical instrument within the lumen. The protrusions may, in some cases, be further configured to direct the fluid flow around the medical instrument. The protrusions can be located at a proximal end, distal end, and/or intermediate portion along the length of the lumen of the accessory.

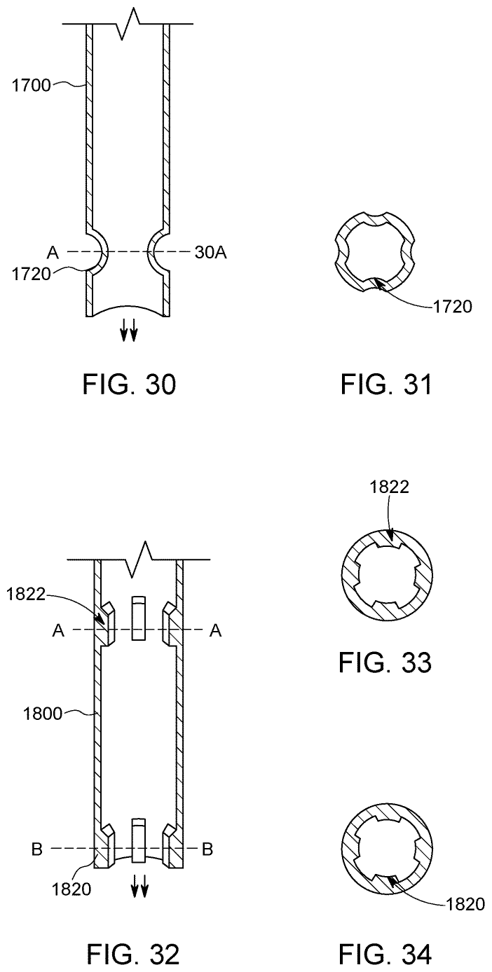

As illustrated in FIG. 30, the protrusions 1720 can be positioned at an open distal end of the medical instrument accessory 1700 and gas can pass between the protrusions 1720 substantially concentrically around the medical instrument (not shown). The protrusions 1720 may be in the form of dimples in and/or on the outer wall, extending the wall into the lumen. FIG. 31 illustrates a cross-section through line A-A of FIG. 30 of the medical instrument accessory 1700. In the example shown, the protrusions 1720 are dimples formed in the wall of the medical instrument accessory 1700, having a convex portion extending into the lumen and a corresponding concave portion on an outer surface of the medical instrument accessory 1700. As shown in FIG. 31, the protrusions 1720 can be spread radially to hold a medical instrument substantially concentrically in the medical instrument accessory 1700 and the gas flow can pass between the protrusions 1720 around the medical instrument. In some cases, the protrusions can be spaced uniformly (or substantially uniformly) around the diameter of the accessory lumen. In other examples, the protrusions can be spaced non-uniformly around the diameter of the accessory lumen, and/or spaced non-uniformly axially along the accessory lumen, creating gas flow paths of different sizes.

In some cases, the at least one guide element can include more than one set of structures (such as ribs, protrusions, fins, dimples, bumps or the like) on the medical instrument accessory. The more than one set of structures can be located at any position, or multiple positions, along the body of the medical instrument accessory. As illustrated in FIG. 32, the guide elements, here shown as ribs 1820, can be positioned at a first location at a distal end of the medical instrument accessory 1800. Ribs 1822 can be positioned in a second location proximal to the first location on the medical instrument accessory 1800. The gas can pass between the ribs 1820 and ribs 1822 substantially concentrically around the medical instrument (not shown).

FIG. 33 illustrates a cross-section through line A-A of FIG. 32 of the medical instrument accessory 1800 through guide elements, shown here as ribs 1822. FIG. 34 illustrates a cross-section through line B-B of FIG. 32. As shown in FIGS. 33 and 34, the ribs 1820 and ribs 1822 can be spread radially at the two locations to hold the medical instrument substantially concentrically in the medical instrument accessory 1800. Gas flow can pass between the ribs 1820 and ribs 1822 around the medical instrument. In some cases, the ribs 1820 and ribs 1822 can be positioned along the accessory at any number of locations any number of times. In some cases, the ribs can be located adjacent the proximal end of the medical instrument accessory.

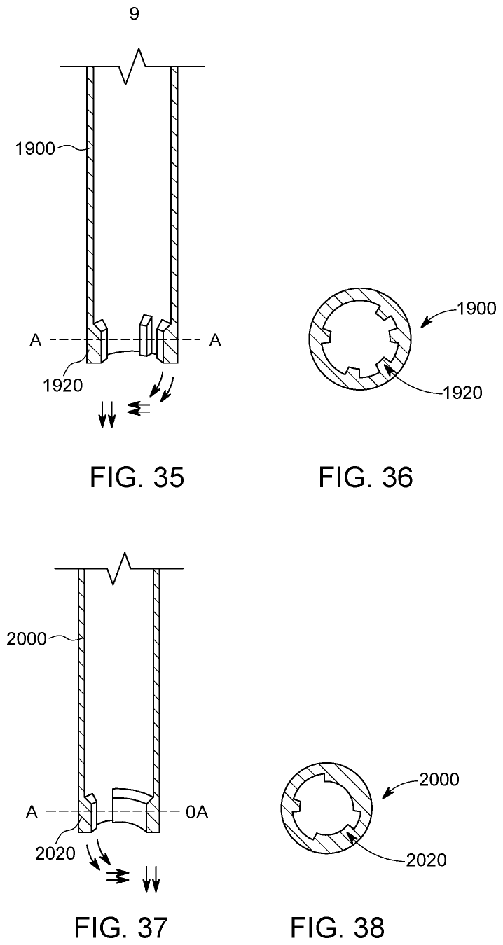

In some cases, the guide elements may be evenly sized and spaced unevenly around the accessory. The guide elements can create a channel of gas flow between adjacent guide elements. FIGS. 35-36 illustrate one example in which the guide elements are provided in the form of ribs 1920. The ribs 1920 can be positioned at the distal end of the medical instrument accessory 1900. The ribs 1920 can be unevenly spaced around the accessory 1900, which may assist to deflect contamination or water droplets away from the viewing portion or encourage evaporation of condensation/fogging that has formed on the viewing portion as illustrated by the arrows shown at the distal end of the accessory 1900 in FIG. 35. The ribs 1920 can be located at any point along the accessory 1900 or the full length of the accessory 1900. The ribs 1920 can be spaced unevenly around the accessory 1900 to direct an entrainment of gas over the medical instrument. Uneven spacing of the ribs 1920 can create different size channels around the inner wall of the accessory 1900. The different sized channels can result in different gas speeds and may create pressure differentials at the distal end of the accessory 1900. FIG. 36 illustrates a cross-section through line A-A of FIG. 35, illustrating the uneven spacing of ribs 1920 around the inner wall of the accessory 1900. The ribs 1920 can hold the medical instrument substantially concentrically within the accessory 1900.

In some cases, the guide elements may be of different sizes, spaced around the accessory. The one or more guide elements can define a channel of gas flow between adjacent guide elements. As illustrated in the example of FIGS. 37-38, the guide elements are shown in the form of ribs 2020, although other forms of guide elements as described herein may also be used. The ribs 2020 can be positioned at the distal end of the medical instrument accessory 2000. The ribs 2020 can have different sizes covering different portions around the circumference of the inner sidewall of medical instrument accessory 2000. The ribs 2020 can be of different sizes and spaced around the accessory 2000 to entrain gas flow over the medical instrument, shown by the arrows at the distal end of the accessory 2000 in FIG. 37. The ribs 2020, can be located at any point along the accessory or the full length of the accessory.

Different size channels can be created by the different width of the guide elements around the inner wall of the accessory. The different sized channels can have different gas speeds and create pressure differentials at the distal end of the device. As illustrated in FIG. 38, which is a cross-section through line A-A of FIG. 37, the ribs 2020 can be of different widths spaced around the inner wall of the accessory and can hold the scope concentrically within the accessory. Although the ribs 2020 are shown at the distal end, the ribs 2020 can be of any length.

The at least one guide element can include one or more notches or channels within the inner wall of the body of the medical instrument accessory to direct gas flow substantially concentrically around the medical instrument. The notch(es) or channel(s) in the body of the medical instrument accessory can define channel(s) for gas flow no matter where the medical instrument is positioned within the accessory. Depending on the axial position of the medical instrument in the accessory, the gas will either flow substantially concentrically around the medical instrument or be entrained over it.

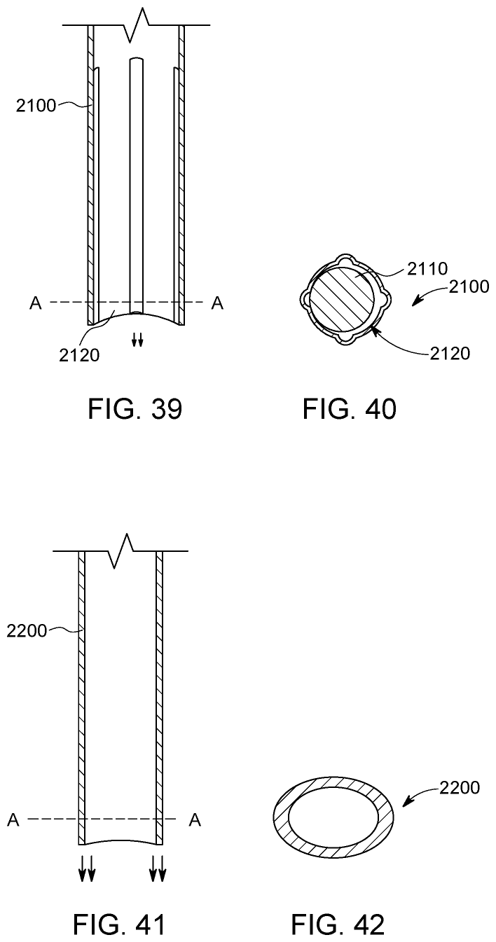

As illustrated in FIGS. 39-40, the notch(es) or channel(s) 2120 can be positioned substantially along the length of the body of the accessory 2100. In other examples, the notch(es) or channel(s) may be positioned at the proximal end, distal end and/or mid-way along the length of the body of the accessory. As illustrated in FIG. 40, which is a cross-section through line A-A of FIG. 39, the notches or channels 2120 can allow gas to continue flowing around the medical instrument 2110 even if the medical instrument 2110 is pressed against inside an inside wall of accessory 2100. The notches or channels 2120 may also allow for gas to continue flowing in cases where the inner diameter of the accessory is similar, or substantially the same as, the outer diameter of the medical instrument. Although the notches or channels 2120 are shown evenly spaced and extending a large portion of the body of the accessory, the notches or channels can be of any length and can be spaced evenly or unevenly. In some cases, the notches or channels can extend the entire length or substantially the entire length of the lumen of the accessory. In other examples, the notches or channels may extend only a partial length of the lumen of the accessory. In some examples, the ends of the notches or channels may be offset from the proximal and/or distal end of the lumen.

The at least one guide element can include a non-circular cross-section in the body of the medical instrument accessory. The medical instrument can have a circular or substantially circular shaft. Therefore, no matter the positioning of the medical instrument within the medical instrument accessory, gas can flow through the accessory. As illustrated in FIGS. 41-42, the medical instrument accessory 2200 can have a shape that is noncircular (for example, ovular or hexagonal) thereby allowing gas flow around the medical instrument. Depending on the axial positioning of the scope, the gas can either be directed around the medical instrument, such as substantially concentrically, or be entrained over the medical instrument. FIG. 42 illustrates a cross-section through line A-A of FIG. 41 of the medical instrument accessory 2200 the body of the medical instrument. As illustrated in FIG. 42, the non-circular shape of the accessory 2200 can allow gas to flow around the medical instrument in any position. Although the non-circular cross-section of the body of the accessory is shown as an oval shape, the non-circular cross-section of the body of the accessory can be any non-circular shape.

### Examples of Flow Controller

The present disclosure provides examples of a flow controller 300 for use with an evacuation line of a surgical system. A surgical system 100 including functionality for venting fluid from a body cavity 2 of a patient (for example as described above with reference to FIGS. 2 and 43) may incorporate any of the example flow controllers disclosed herein.

Suction may be required during a surgical procedure to evacuate fluids, smoke and/or debris from a body cavity. Evacuation may be provided via an evacuation line, as described above. Such evacuation lines may be connected to a suction source providing constant suction. Suction sources may include discrete suction units and/or hospital wall ports. Valves may be used in conjunction with the evacuation line/suction source.

It may be desirable to control the amount/extent of evacuation, or to provide finer control over the amount/extent of evacuation. Further, in some instances, it may be desirable to switch the evacuation “OFF”. It may be desirable to control evacuation flow from within the surgical sterile field, including proximal to the patient. The example flow controllers 300, 3300 as described herein may be operable to control fluid flow in a fluid evacuation line, such as fluid evacuation line 23 in the surgical system 100 of FIG. 43.

The example flow controllers disclosed herein may be retro-fitted to existing surgical systems including an evacuation line. For example, the flow controllers disclosed herein may be joined in fluid connection with an existing evacuation line to enable control of fluid flow in the evacuation line.

The flow controller can be single use (disposable) or reusable. Alternatively, parts of the flow controller can be single use (disposable) or reusable. The flow controller may be made of materials that are sterilisable.

The flow controller may include a body having a first body portion configured for fluid communication with a first portion of the fluid evacuation line 23 and a second body portion configured for fluid communication with a second portion of the fluid evacuation line 23. A fluid flow path is defined between the first portion of the fluid evacuation line 23 and the second portion of the fluid evacuation line 23.

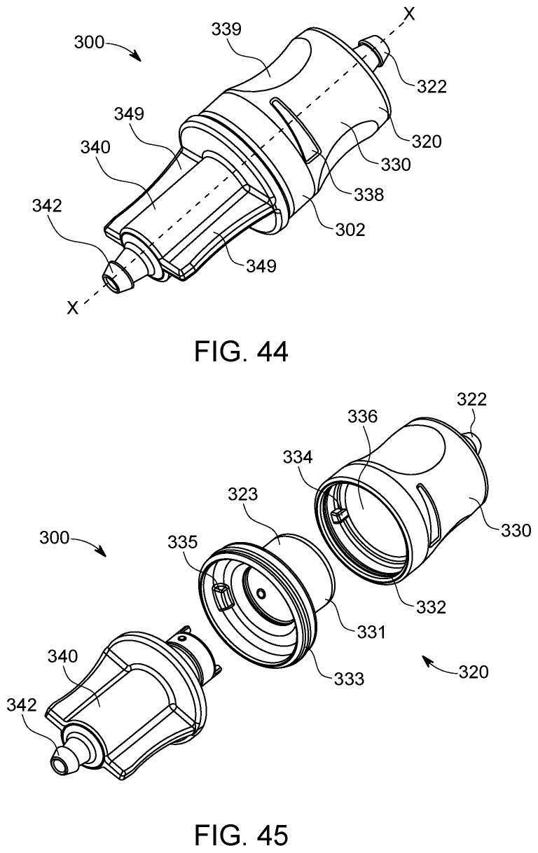

An example flow controller 300 is shown in FIG. 44. The flow controller 300 comprises a body 302, comprising a first body portion 320 coupled with a second body portion 340. The first body portion 320 comprises a first fluid connecting portion 322 configured for connection with the first portion of the fluid evacuation line 23. The second body portion 340 comprises a second fluid connecting portion 342 configured for connection with the second portion of the fluid evacuation line 23. A fluid flow path is defined between the first fluid connecting portion 322 and the second fluid connecting portion 342.

In the illustrated examples the second fluid connecting portion 342 comprises a fluid inlet, for connection with an upstream portion of the fluid evacuation line 23 (that is, closest to the body cavity 2). The first fluid connecting portion 322 comprises a fluid outlet, for connection with a downstream portion of the fluid evacuation line 23 (that is, further from the body cavity 2). However, it will be appreciated that the flow controller 300 may be connectable with the fluid evacuation line 23 in the reverse orientation.

The first and second fluid connecting portions 322, 342 may be provided in axial alignment with each other. As indicated in FIG. 44, the first and second fluid connecting portions 322, 342 may be coincident with a longitudinal axis X of the body 302. The first and second fluid connecting portions may be configured for connection in fluid communication and in line with the upstream and downstream portions of the fluid evacuation line 23.

Providing the first and second fluid connecting portions 322, 342 in line may assist fitting the flow controller 300 into an existing (or new) evacuation line 23. For example, providing the first and second fluid connecting portions 322, 342 in axial alignment with each other may assist in minimising a size of the flow controller 300 and/or enhancing ease of use of the flow controller 300. Further, providing the first and second fluid connecting portions 322, 342 in axial alignment with each other may reduce the chance of liquid becoming trapped in the flow controller. There may be a substantially direct line for the fluid (gas or otherwise) to pass through, for example with minimal internal features of the flow controller that may trap liquid.

The second body portion 340 is movable relative to the first body portion 320 (or vice versa) to selectively open, close and/or vary the fluid flow path between the first fluid connecting portion 322 and the second fluid connecting portion 342.

As described herein, the fluid flow path being closed may be understood to mean fluid flow through the fluid flow path being inhibited and/or substantially prevented. For example, when the fluid flow path is closed, fluid flow through the fluid flow path may be obstructed or substantially blocked. The fluid flow path being open, or at least partially open, should be understood to mean that fluid flow through the fluid flow path is allowed. The fluid flow path may be varied, for example by varying a degree of obstruction of the fluid flow through the fluid flow path. Varying the fluid flow path may vary one or more properties of the fluid flow, such as a rate (e.g. a volumetric flow rate), velocity and/or pressure of the fluid flow.

In some examples the flow controller 300 may comprise an intermediate portion 331, positioned between the first body portion 320 and the second body portion 340. The intermediate portion 331 may be part of the first body portion 320 or the second body portion 340. The intermediate portion 331 may comprise a fluid flow opening. For example, where the intermediate portion 331 is part of the first body portion, the intermediate portion may comprise the first opening 325. Where the intermediate portion 331 is part of the second body portion 340, the intermediate portion 331 may comprise the second opening 340. Where the intermediate portion 331 is separate from either of the first or second body portions 320, 340, the intermediate portion 331 may comprise either of the first opening 325 or the second opening 345. The intermediate portion 331 may be configured to be received in one of the first body portion 320 and the second body portion 340. The intermediate portion 331 may be configured to receive the other of the first body portion 320 and the second body portion 340.

FIG. 45 shows components of an example flow controller 300 in a disassembled state. In the illustrated examples, the first body portion 320 comprises a housing 330 and an intermediate portion in the form of an insert 331. The insert 331 and the housing 330 may be assembled to form the first body portion 320. For example, the insert 331 may be at least partially receivable within the housing 330. In other examples, the first body portion 320 may be formed in a single piece. For example, the housing 330 and the insert 331 may be integrally formed. The fluid connecting portion 322 may be provided on the housing 330. In some examples, as described above, the second body portion 340 may comprise a housing and the insert 331. Where the second body portion 340 comprises the insert 331, a housing of the second body portion 410 may be integrally formed with the insert 331.

As shown in FIG. 45, for example, the housing 330 may define an internal portion 336. The internal portion 336 is in fluid communication with the fluid connecting portion 322. The insert 331 may be configured to be at least partially received within the internal portion 336 of the housing 330. The insert 331 and housing 330 may be configured for assembly in a predetermined alignment relative to each other.

When assembled, the insert 331 is rotationally and axially fixed relative to the housing 330. The insert 331 and/or housing 330 may include one or more features configured to inhibit movement between the insert 331 and the housing 330. For example, one or both of the housing 330 and the insert 331 may include at least one protrusion configured to be received in at least one corresponding recess of the other of the housing 300 and the insert 331.

The housing 330 and insert 331 may be configured for assembly with each other via locking engagement. For example, the housing 330 and insert 331may be configured for a snap-locking engagement. The locking engagement may be configured such that, in use, the insert 331 is not readily separable from the housing 330. In other examples, the insert 331 and housing 330 may be configured for disassembly and re-assembly (for example, via a releasable snap-fit engagement as opposed to a locking engagement).

In the example flow controller of FIG. 45, the insert 331 includes a protrusion in the form of a ridge 333. The ridge 333 is configured to be received in a recess in the form of a corresponding groove 332 in the housing 330 to inhibit axial movement of the insert 331 relative to the housing 330. In other examples, the insert 331 may comprise an annular flange configured to abut an edge of the housing 300. A gap in the annular flange may correspond with a protrusion on the edge of housing 300 to limit relative rotation between the insert 331 and the housing 300. In other examples, the engagement between the insert 331 and the housing 300 may be provided by a friction fit, a taper fit, or by a ridge on one of the insert 331 and the housing 300 and a taper on the other of the insert 331 and the housing. In other examples, the insert 331 and housing may be fixed by other means, such as by a weld, adhesive, or by a retaining element such as a clip or fastener.

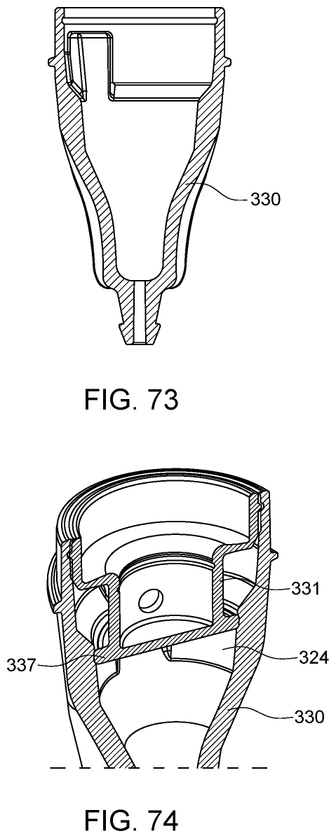

In the example of FIG. 73, the housing 330 has a wall defining a tapered inner surface 3301. As shown in FIG. 74, the insert 331 may include a lip 337. The lip 337 may be configured to abut the inner surface of the housing 330, for example at ridge 324 on the internal surface of the housing. This may provide a friction and/or interference fit between the housing 330 and insert 331, which may inhibit separation of the housing 330 and insert 331.

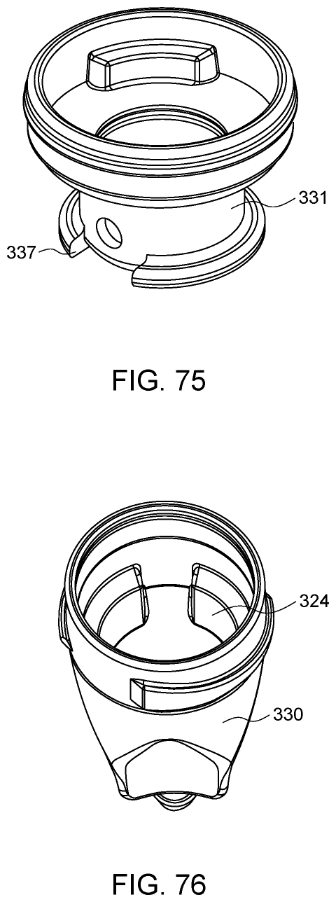

As shown in FIGS. 75 and 76, the insert 331 includes a cut-out 337a in the lip 337 and the housing includes a cut-out 324a formed through the ridge 324. The insert 331 may be configured to be received in the housing 330 with the cut-out 327a in the lip 337 aligned with the cut-out 324a in the ridge 324, such that a channel is provided through the lip 337 and ridge 324 to provide a fluid flow path therethrough. The channel may facilitate directing of fluid flow through the flow controller 300, particularly when coupled with the tapered inner surface of the housing 330. This may aid in minimising trapping of any fluid within the fluid controller.

The housing 330 and/or insert 331 may include one or more rotation inhibiting elements for inhibiting relative rotation of the housing and the insert. For example, the insert 331 and/or the housing 330 may include one or more projections receivable in a corresponding one or more recesses of the other of the insert 331 or the housing 330 to inhibit relative rotation between these parts. In the example shown in FIG. 45, the rotation inhibiting element comprises a slot 335 on the insert 331 and a protrusion 334 on the housing 330. The protrusion 334 is receivable in the slot 335 to inhibit rotation of the insert 331 relative to the housing 330. In other examples, the slot 335 may be in the housing 330 and the protrusion 334 may be on the insert 331.

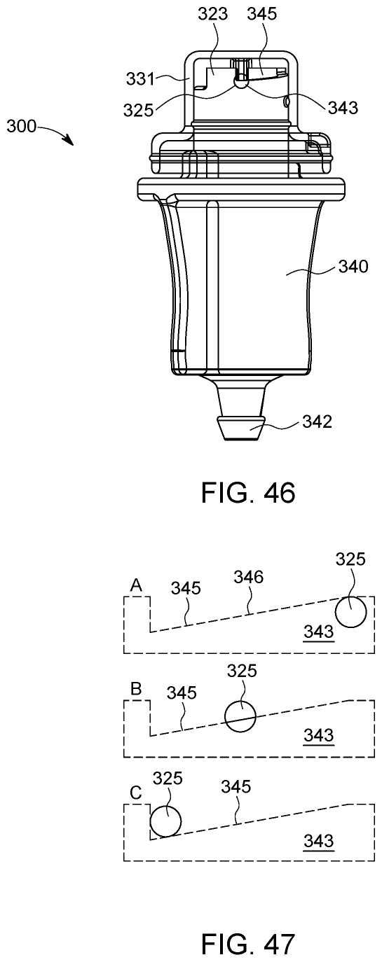

FIG. 46 shows a partially transparent view of the flow controller 300 of FIG. 44 (with the housing 330 not shown to enhance visibility of inner features of the first body portion 320). As can be seen in FIG. 46, the first body portion 320 defines a first opening 325. The first opening 325 is in fluid connection with the first fluid connecting portion 322 (not shown in FIG. 46). The second body portion 340 defines a second opening 345 in fluid connection with the second fluid connecting portion 342. The first body portion 320 is movable relative to the second body portion 340 to bring the first opening 325 into or out of fluid communication with the second opening 345, thereby to selectively open, close and/or vary the fluid flow path through the first and second openings 325, 345.

The first and/or second body portions 320, 340 can be movable to bring the first and second openings 325, 345 into or out of at least partial overlap with each other to vary the fluid flow path. That is, the flow controller 300 may be configured such that the fluid flow path is closed when the first and second openings 325, 345 are out of alignment with each other and the fluid flow path is at least partially open when the first and second openings 325, 345 are in at least partial alignment with each other. The first and/or second body portions 320, 340 may be movable between a first position in which the first and second openings 325, 345 do not overlap with each other and a second position in which the openings 325, 345 are at least partially overlapping with each other. The degree of overlap between the openings 325, 345 may vary as the first and second body portions 320, 340 are moved between the first position and the second position.

In some examples, the movement between the first and second body portions 320, 340 may be a rotational movement. In such examples, the first body portion 320 and the second body portion 340 may be axially fixed relative to one another. In other examples, the movement between the first and second body portions 320, 340 may be an axial movement. In other examples, the movement may be a movement in more than one direction, and/or involving rotation and/or axial movement between the first and second body portions 320, 340.

In the illustrated examples, the movement between the first and second body portions 320, 340 is a rotational movement about a longitudinal axis X of the body. As such, in the illustrated examples, the first position is a first angular position and the second position is a second angular position, relative to the longitudinal axis X. The movement between the first and second body positions may be within a predefined angular range of motion. The angular range of motion may be between about 90 degrees and about 120 degrees, or any value there between, for example.

The first and second openings 325, 345 may be provided in respective wall portions of the first and second body portions 320, 340. For example, the openings 325, 345 may extend through respective side-wall and/or end-wall portions of the first and second body portions 320, 340. For example, the first body portion 320 of the flow controller 300 includes a cylindrical side-wall 323. Similarly, the second body portion 340 comprises a cylindrical side-wall 343.

In the examples shown in FIGS. 45 and 46, the wall 323 of the first body portion 320 is part of the insert 331. The wall 323 may include a side wall portion and an end wall portion. When the flow controller 300 is assembled, the wall 323 is located within the internal portion 336 of the housing 330. The wall 323 of the insert 331 may substantially close the internal portion 336. The wall 323 of the insert 331 of the first body portion 320 may comprise the first opening 325. The first opening 325 may extend through the wall 323, such that the fluid flow path extends through the insert 331 and through the internal portion 336 of the housing 330 to the fluid connecting portion 322.

The wall 343 of the second body portion 340 defines an inner portion (or cavity) in fluid communication with the second fluid connecting portion 342. The inner portion of the second body portion 340 may have a perimeter less than a perimeter of the inner portion 336 of the housing 330. The wall 343 of the second body portion 340 may comprise the second opening 345. In the illustrated example, the second opening 345 extends through a thickness of the wall 343.

In some examples, the walls 323 and 343 may comprise substantially cylindrical side-walls. However, other shapes for the walls are also contemplated. At least a portion of one of the cylindrical side-walls may be received within the other cylindrical side wall. At least a portion of the inner portion of the second body portion 340 may be received within the first body portion 320. In other examples, the reverse configuration is contemplated. That is, a portion of the first body 320 portion may be received within the second body portion 340.

In the illustrated examples, the cylindrical side-wall 343 of the second body portion 340 is partially received within the cylindrical side-wall 323 of the first body portion 320, such that the first and second body portions 320, 340 are partially nested relative to one another. For example, as best shown in FIG. 46 the cylindrical side wall 343 of the second body portion 340 is partially received within the cylindrical side wall 323 of the insert 331. As such, the inner portion defined by the wall 343 of the second body portion is partially received within the inner portion defined by the wall 323 of the insert 331.

The walls 323, 343 of the first and second body portions 320, 340 may be slidably engaged with each other. The movement between the first and second body portions 320, 340 may include a sliding movement of the wall 323 of the first body portion against the wall 343 of the second body portion. In the first position, the first opening 325 may be obstructed by the wall 343 of the second body portion 340 to inhibit fluid flow therethrough. In some examples, in the second position, the first opening 325 may be substantially unobstructed to allow fluid flow therethrough. In some examples, the first opening 325 may be partially obstructed in the second position. In the illustrated examples, the walls 323, 343 are configured to slide against one another as the second body portion 340 is rotated relative to the first body portion 320.

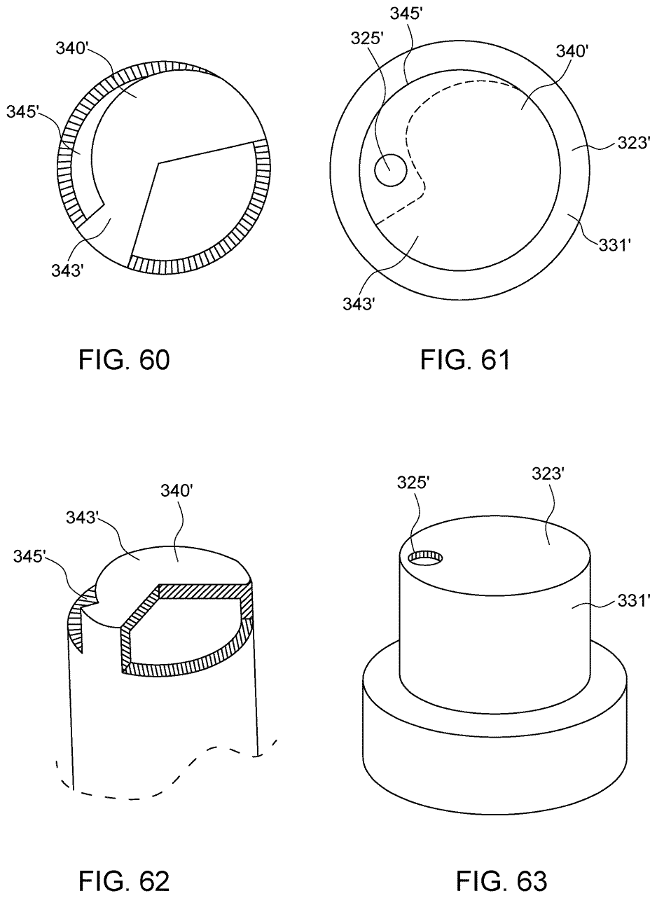

In the example flow controller 300, the first opening 325 extends through a side-wall portion of the wall 323 of the first body portion 320 and the second opening 345 extends through a side-wall portion of the wall 343 of the second body portion 340. In some examples, the first and second openings 325, 345 may extend additionally and/or alternatively through an end-wall portion of the first and second body portions 320, 340, respectively. For example, as shown in the example configuration in FIGS. 60-63, the first opening 325′ extends through the end-wall portion of the wall 323′ of the insert 331′, while the second opening 345′ extends through both the side-wall portion and end-wall portion of the wall 343′ of the second body portion 340′. As such, the first and second openings 325′, 345′ are in-line with the longitudinal axis of the flow controller. In this example, relative rotation of the second body portion 340′ and/or first body portion 320′ with respect to each other changes the relative positions of the first and second openings 325′, 345′ to vary an area of the first opening 325′ which is obstructed by the end-wall portion of the wall 343′ of the second body portion 340′.

In some examples, a length of the second opening 345 may extend at least partially in the direction of the movement between the first and second body portions 320, 340. For example, in the illustrated examples, the second opening 345 has a length extending in the rotational direction. As a further example, where the movement between the first and second body portions 320, 340 is in the axial direction, the length of the second opening may extend at least partially in the axial direction. A secondary dimension (such as a height or a width, for example) of the second opening 345 may vary along the length of the second opening 340.

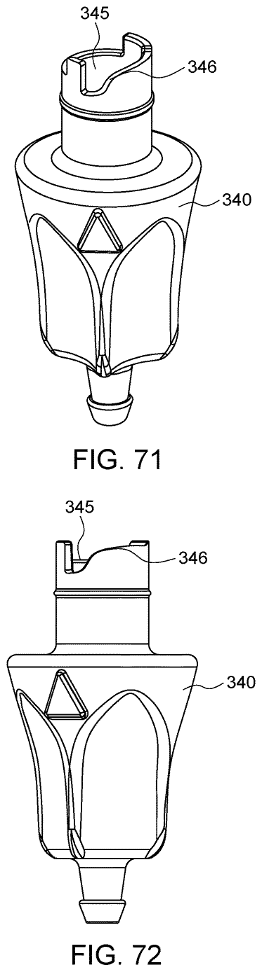

For example, the second opening 345 may taper along its length. In some examples, the second opening 345 may taper substantially linearly. In other examples, the second opening 345 may vary non-linearly, such as shown in FIGS. 71 and 72. However, other manners of variation of the secondary dimension of the second opening 345 are also contemplated. In some examples, the second opening 345 may be discontinuous, such as comprising a series of apertures, which may have varying sizes. For example, the second opening 345 may comprise a series of apertures of gradually increasing or decreasing size.

In the examples of FIGS. 46 to 50, the second opening 345 is defined by a notch in an end of the wall 343 of the second body portion 340. The second opening 345 has a ramped base portion 346, as best shown in FIG. 47, such that the second opening 345 has a shape of an inverted right-angled triangle. As illustrated, the second opening 345 adjoins an open end of the second body portion 340, such that the notch defines an open shape. However, in other examples, the second opening 345 may comprise a “cut-out” or “punch-out”, surrounded by material of the wall 343 on all sides to define a closed shape. Alternative shapes for the second opening 345 are also contemplated. For example, the second opening 345 may include alternative angles and/or curved portions such as a curved base, sides and/or corners. For example, FIGS. 71 and 72 show a second opening 345 having a base portion 346 that is curved in an “S” shape, and curved corner regions.

The first opening 325 may have at least one dimension shorter than the length of the second opening 345. The first and second body portions 320, 340 may be movable relative to each other to align the first opening 325 with a selected region of the second opening. A degree of obstruction of the first opening 325 by the wall 343 of the second body portion 320 may be variable depending on a region of the second opening 345 with which the first opening 325 is aligned. In the illustrated examples, the first opening 325 has a circular shape. A diameter of the first opening 325 is smaller than the length of the second opening 345, and equal to or smaller than a maximum height of the second opening 345. However, other shapes for the first opening 325 are also contemplated.

FIG. 47 illustrates, diagrammatically, the relative alignments of the first and second openings 325, 345 in the first position (FIG. 47-A), the second position (FIG. 47-C) and in an intermediate position, between the first and second positions (FIG. 47-B) , according to one example of the present disclosure.

In the first position (FIG. 47-A), the first opening 325 is positioned out of alignment with the second opening 345. That is, there is no overlap between the open areas of the first and second openings 325, 345. The first opening 325 is therefore substantially blocked by the wall 343 of the second body portion to substantially prevent fluid flow therethrough. In this position, the fluid flow path is closed.

In the second position (FIG. 47-C), the first opening 325 is aligned with the second opening 345 such that the first opening 325 is substantially unobstructed by the wall 343 of the second body portion 340. The first opening 325 is therefore substantially unblocked or unimpeded by the wall 343 of the second body portion to permit fluid flow therethrough.

FIG. 47-B shows an intermediate position between the first position and the second position. In this position, the first opening 325 is only partially obstructed by the wall 343 of the second body portion 340. That is, there is a partial overlap between the open areas of the first opening 325 and the second opening 345. This partial obstruction of the first opening 325 serves to partially restrict fluid flow therethrough. The area of the first opening 325 that is obstructed by the wall 343 of the second body portion 340 may vary as the second body portion 340 is moved between the first position and the second position. In some examples, the variation in obstructed area of the first opening 325 may be substantially linear between a minimum obstruction and a maximum obstruction. In other examples, such as the example of FIG. 47, the variation is non-linear.

One or more parameters of fluid flow through the fluid flow path may be variable depending on the region of the second opening 345 with which the first opening 325 is aligned. For example, varying the obstructed area of the first opening 325 may vary the flow rate though the fluid flow path.

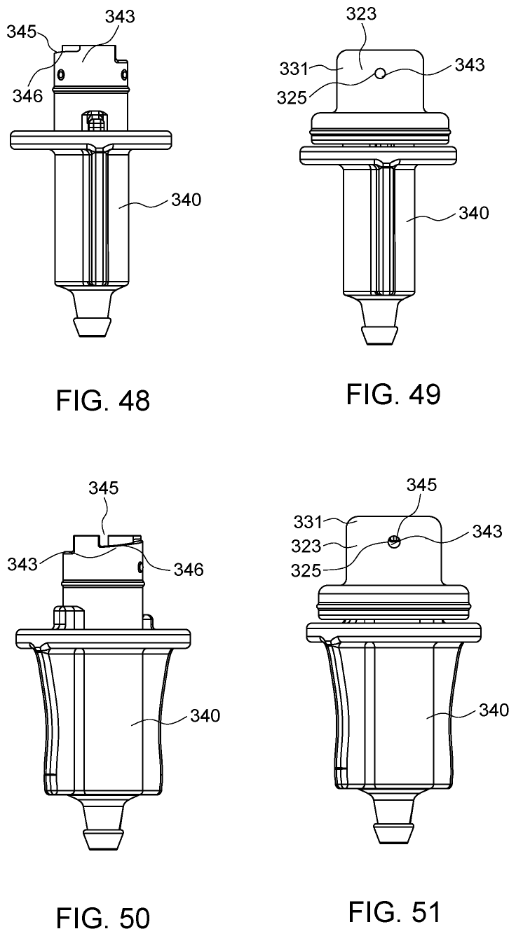

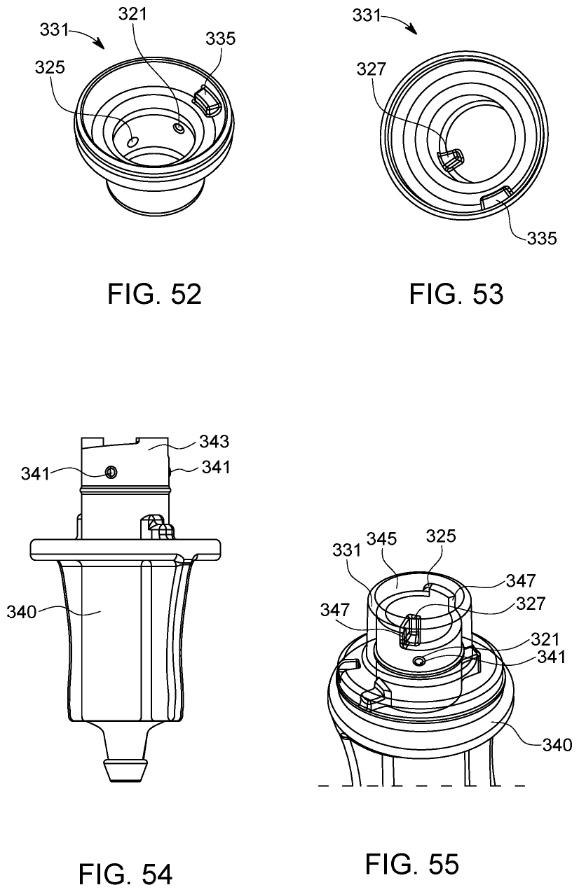

FIGS. 48 and 49 show the second body portion 340 in the first angular position, with and without the insert 331, respectively. As can be seen in FIG. 49, in the first position, the wall 343 of the second body portion 340 obstructs the first opening 325 to close the fluid flow path. FIGS. 50 and 51 show the second body portion 340 in the second angular position. In this example (and as shown in FIG. 46) in the second position the wall 343 still partially obstructs the first opening 325. In other examples, however, the first opening 325 may be substantially unobstructed in the second position, as shown in FIG. 47-C, for example.

In some examples, the flow rate may be variable between a minimum flow rate and a maximum flow rate. The minimum flow rate may be about 0 litres per minute. The maximum flow rate may be about 3 litres per minute, about 4 litres per minute, about 5 litres per minute, about 6 litres per minute, about 7 litres per minute, about 8 litres per minute, about 9 litres per minute, about 10 litres per minute, up to about 15 litres per minute or more. The maximum flow rate may be limited, at least partially, by requirements to maintain the body cavity pressure and/or volume within workable and safe limits, and/or due to resistance to flow within system components, such as tubing sizing, for example. The minimum flow rate may be defined at the first position and the maximum flow rate may be defined at the second position. However, in other examples, a maximum flow rate may be defined at an intermediate position. However, other flow variation profiles are also contemplated. For example, depending on the shape of the first and second openings 325, 345, the flow controller could be configured such that moving the second body portion 340 relative to the first body portion 320 causes the flow rate to change rapidly from a minimum flow rate (e.g. substantially no flow) to a maximum flow rate and then gradually reduce thereafter.

The flow controller 300 may include a movement limiter, configured to limit the relative movement between the first and second body portions 320, 340. The movement limiter may be configured to limit the movement to between limits defined at the first position and the second position, for example.

The movement limiter may comprise a protrusion 327 on one of the first body portion 320 or the second body portion 340 and at least one stop 347 on the other of the first body portion or the second body portion. The protrusion 327 may be configured to abut the at least one stop 347 in the first position and the second position to inhibit movement beyond said positions.

In the illustrated examples, the movement limiter is a rotation limiter, configured to limit the relative rotation between the first and second body portions 320, 340 to between the first and second angular positions. The rotation limiter comprises a protrusion 327. The first body portion 330 comprises a protrusion 327 and the second body second body portion 340 comprises a pair of stops 347. In this example, the protrusion 327 is located on the insert 331, but may be located elsewhere on the first body portion 330. The protrusion 327 is configured to engage one of the pair of stops 347 in the first position and the other of the pair of stops 347 in the second position. This inhibits rotation of the second body portion 340 beyond the first position or the second position, and defines the movement of the first opening 325 relative to the second opening 345.

The flow controller may include one or more grip elements for facilitating manual rotation of the second body portion 340 relative to the first body portion 320 by a user. One or both of the first and second body portions 320, 340 may include one or more grip elements.

Where both the first and second body portions 320, 340 include grip elements, the grip elements may be the same, similar, or may be different. In some examples, the first body portion 320 may comprise differently shaped grip elements to the second body portion 340, for example to encourage a user to rotate either the first body portion 320 or second body portion 340 with respect to the other.

As best seen in FIG. 44, in some examples, the second body portion 340 includes a pair of fins 349, which extend from the second body portion 340. The fins 349 may facilitate application of rotational force to the second body portion 340 by a user. In other examples, the flow controller 300 may include a single fin 349 or more than two fins 349. In the illustrated example, the fins 349 are positioned opposite each other about the longitudinal axis X and extend substantially symmetrically about the longitudinal axis X. However, in other examples, the fins 349 may be dissymmetrical about the longitudinal axis X and/or not positioned opposite each other.

The first body portion 320 may also include one or more grip portions. Referring to FIG. 44, the first body portion 320 includes a pair of recessed regions 339 configured for facilitating a user's grip. The recessed regions 339 are positioned opposite each other about the longitudinal axis X. However, other positioning for the recessed regions 339 is also possible.

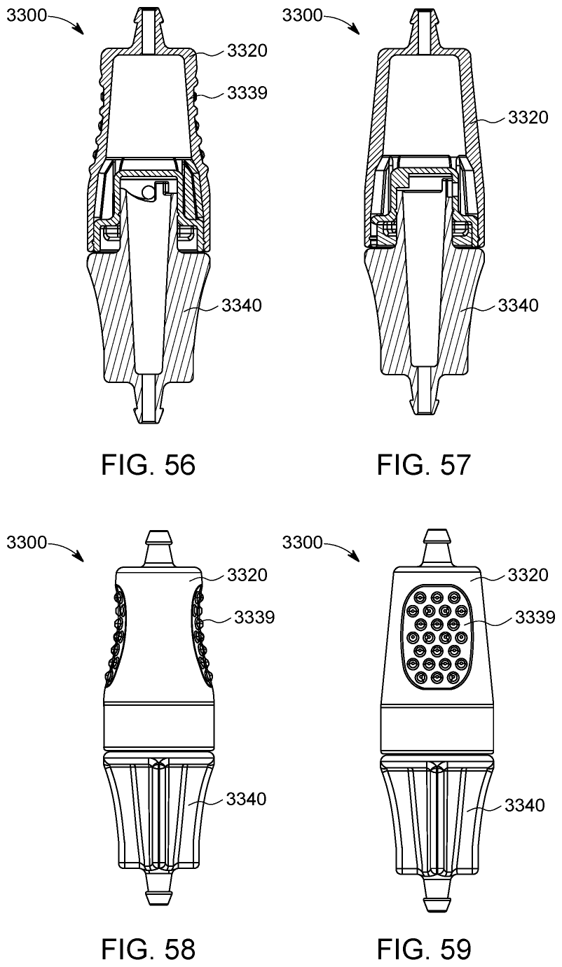

Additionally or alternatively, one or more grip portions may include surface features configured to increase friction between the grip portion and the users hand. For example, a further example flow controller 3300 is shown in FIGS. 56-59. The flow controller 3300 may share one or more features of the flow controller 300 as described herein. The flow controller 3300 comprises a first body portion 3320 and a second body portion 3340. The first body portion 3320 may include grip portions. For example, as best shown in FIG. 59, the first body portion 3320 includes grip portions 3339 including a textured grip surface. The grip portions 3339 may be recessed or not. As best shown in FIG. 59, the grip portions 3339 each include a plurality of projections in a patterned arrangement. The grip portions 3339 may enhance a user's grip on the first body portion 3320 when turning the second body portion 3340 relative to the first body portion 3320.

The flow controller 300 may comprise one or more indicators for indicating a relative position of the first and second body portions 320, 340. The indicators may include visual, audio, tactile and/or haptic indicators. In some examples, the indicators may include indicia on one or both of the first and second body portions. For example, the flow controller 300 may include a first indicium on the first body portion 320 and a second indicium on the second body portion 340. The indicia may be configured such that a relative alignment of the first and second indicia provides a visual indication of a position of the first opening relative to the second opening. As shown in FIG. 44, the first body portion 320 includes a flow indicator 338 on an outer surface of the housing 330. A relative alignment between the nearest fin 349 of the second body portion 340 and the flow indicator 338 provides a visual indication of the position of the first opening relative to the second opening, and of a flow rate through the fluid flow path.

In other examples, the first body portion 320 may also comprise one or more fins. In such examples, a relative alignment between at least one of the fins 349 of the second body portion 340 and at least one of the fins of the first body portion may provide a visual indication of a position of the first opening 325 relative to the second opening 345.

In some examples, the flow controller 300 may comprise one or more retaining elements, configured to retain the first and second body portions in relative alignment in one or more predefined positions. The one or more predefined positions may include the first position, the second position and/or one or more intermediate positions between the first position and the second positions. In some examples, the flow controller 300 comprises a plurality of retaining elements configured to retain the first and second body portions in relative alignment at a corresponding plurality of positions.

The retaining element may be configured to provide frictional resistance to relative movement between the first and second body positions, for example. The frictional resistance may be overcome by application of force (such as rotational force) between the first and second body portions 320, 340 by a user.

The flow controller 300 may include one or more protrusions 341 on one of the first body portion 320 and the second body portion 340, the one or more protrusions 341 receivable in at least one recess on the other of the first body portion 320 and the second body portion 340 to provide resistance to movement between the first body portion 320 and the second body portion 340.

The one or more protrusions and/or recesses may be arranged such that a protrusion is received in a recess at one or more predefined positions of the second body portion 340 relative to the first body portion 320. For example, as shown in FIG. 54, the second body portion 340 includes a pair of protrusions 341 extending outwardly from an outer surface of the cylindrical wall 343. The protrusions 341 are configured to be received in a corresponding recess 321 in the insert 331. However, the reverse configuration of recess and protrusions is also contemplated. The protrusions 341 are positioned to engage the recess 321 when the second body portion 340 is in the first position or the second position relative to the first body portion 320. Other examples may include additional protrusions for positioning the second body portion 340 at one or more intermediate positions between the first and second positions. In other examples, the flow controller may comprise a single protrusion engageable with a plurality of recesses to retain the second body portion 340 in the first and second positions and/or any intermediate positions.

Flow controllers as described herein may be devoid of any filters within the body of the flow controller. The flow controllers 300, 3300 of the illustrated examples do not include any filters or filter material. In such examples, a narrowest passage of the fluid flow path may be defined by the first opening 325 and/or the second opening 345 (rather than by filter material, for example). In some examples, a system including a flow controller 300, 3300 as described herein (such as the system 1 described above) may include one or more filters, such as filter 25, separate to the flow controller 300. As shown in the surgical system 100 of FIG. 43, the filter 25 may be separated from the flow controller 300 by a length of the tubing of the evacuation line 23. The filter 25 may be located outside the surgical sterile zone. This may minimise the size of the flow controller and aid in minimising clutter in the sterile zone.

### Examples of Fluid Line Connector

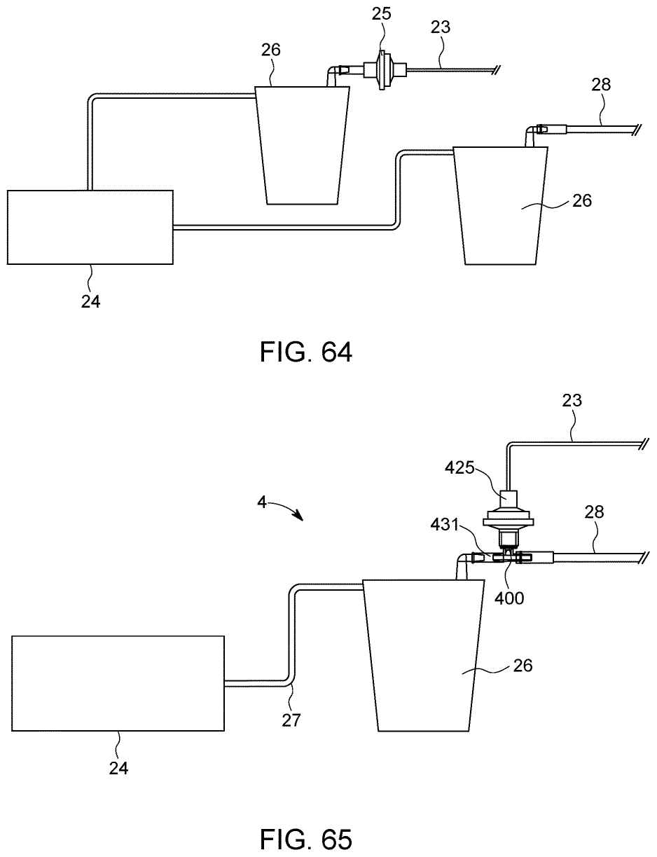

Some surgical procedures may require suctioning of surgical fluids (such as irrigation fluid) from the body cavity 2. This may be in addition to evacuation of surgical gases and/or smoke from the body cavity 2. This may require two evacuation lines: a first fluid evacuation line 23 configured for venting of gases during a surgical procedure (including smoke and/or particulate matter clearance, for example) and a second fluid evacuation line 28 for evacuation of irrigation fluids during the procedure. In conventional systems, each evacuation line 23, 28 is connected to a separate suction canister 26, for example as shown in FIG. 64. The canisters 26 may be connected to a suction source 24, such as a vacuum or wall suction port.

Examples of the present disclosure provide a combination gas/smoke and irrigation fluid evacuation system. One example of a fluid evacuation system 4 is shown in FIG. 65.

The fluid evacuation system 4 is configured for fluidly joining a first fluid evacuation line 23 and a second fluid evacuation line 28 to a common suction canister 26, which is in fluid connection with a suction source 24 (such as a suction pump, wall port) via a suction line 27.

The fluid evacuation system 4 may include a fluid line connector 400, as shown in FIG. 65, for example.

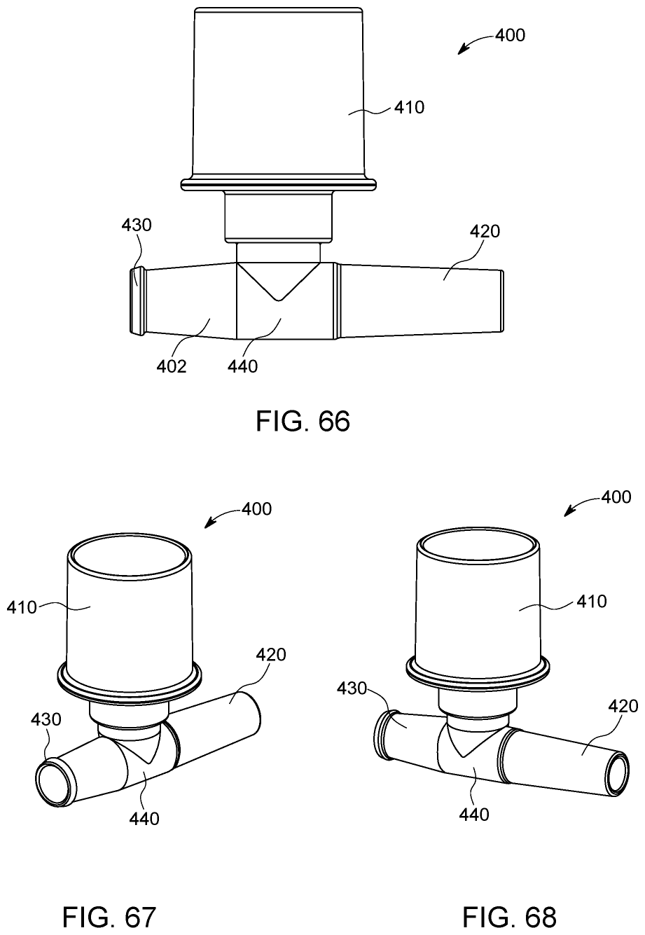

In the example shown in FIGS. 66-70, the fluid line connector 400 comprises a connector body 402. The body 402 comprises a first inlet port 410 for fluid connection with the first fluid evacuation line 23 and a second inlet port 420 for fluid connection with the second fluid evacuation line 28. The body 402 further comprises an outlet port 430 for fluid connection with the suction source 26. The inlet ports 410, 420 and outlet port 430 are fluidly connected by a junction region 440.

In some examples, a plug or cap may be removably coupled to the second inlet port 420 when the second evacuation line 28 is not present or not in use. In some examples, a connector 431 may be connectable to the outlet port 430, as shown in FIG. 65, for example. The connector 431 may be configured to be compatible with a port on the suction canister 26.

Provision of a fluid line connector 400 within the surgical system 100 integrates the irrigation fluid evacuation line 28 and a smoke evacuation line 23 into one attachment that can connect to a single suction canister 26. This may reduce cost, by reducing the number of suction canisters required, and/or may facilitate using system 100 in conjunction with conventional single canister irrigation setups.

In the illustrated example, the first inlet port 410 is substantially perpendicular to the second inlet port 420 and the outlet port 430, which are in axial alignment with each other.

However, other angles between the ports 410, 420, 430 are also possible. The connector 400 may be configured to be used in the orientation as shown in FIGS. 65 to 70, with the first inlet port 410 positioned above the second inlet port 420 and the outlet port 430. However, other configurations and positioning for the first and second inlet ports 410, 420 and the outlet port 430 are also contemplated. For example, where the fluid line connector 400 includes one or more valves, the relative orientation of the ports 410, 420, 430 may be less significant.

In some situations, irrigation fluid may inadvertently enter the first evacuation line 23 at junction 440. This may occur due to a pressure difference created in the fluid connection system 4 when the second evacuation line 28 is activated, and when a flow rate of fluid through the first evacuation line 23 is at zero or under a threshold (e.g. 0.5 LPM).

When no suction is being applied, the pressure throughout the first evacuation line 23, second evacuation line 28, suction canister 26 and suction line 27 (between the suction canister and suction source) may be approximately equal to the pressure of the vacuum applied (in equilibrium). However, when the second evacuation line 28 is activated/opened (for example, to apply suction), the pressure in the second evacuation line 28 becomes similar to atmospheric pressure. For a period of time, the pressure in the first evacuation line 23 and suction line 27 is still held at vacuum. Therefore, fluid in the second evacuation line 28 may be drawn into both the suction canister 26 and/or undesirably into the first evacuation line 23.

The pressures within the system 4 then stabilise after a period of time, such that the pressure differential between the second evacuation line 28 and suction line 27 is greater than between the filter 425 and suction line 27, such that fluid flows towards the suction canister as opposed to through the first evacuation line 23 and/or filter 425. However, this problem may recur when the second evacuation line 28 is deactivated, and then reactivated again, for example if a user is pulsing the second evacuation line 28.

In addition, the physical orientation of the first evacuation line 23 may result in irrigation fluid entering the first evacuation line 23. For example, if the first evacuation line 23 is oriented below the second evacuation line 28, fluid can undesirably pool or pass into the first evacuation line 23 under the influence of gravity.

Fluid entering the first evacuation line 23 may wholly or partially obstruct the evacuation line 23, affecting flow through the first evacuation line 23. Additionally or alternatively, fluid may clog filters (such as filter 425) in connection with the first evacuation line 23. In some cases, the fluid may flow along the evacuation line 23 and back into the body cavity 2, which may increase the risk of introducing contamination to the body cavity.

To avoid or ameliorate one or more of problems above, in some examples, a one-way valve 450 may be positioned in connection with the first evacuation line 23 of the fluid connection system 4, to inhibit fluid from entering the first evacuation line 23. Any filter(s) in connection with the fluid evacuation line 23 may be positioned upstream of the one-way valve 450.

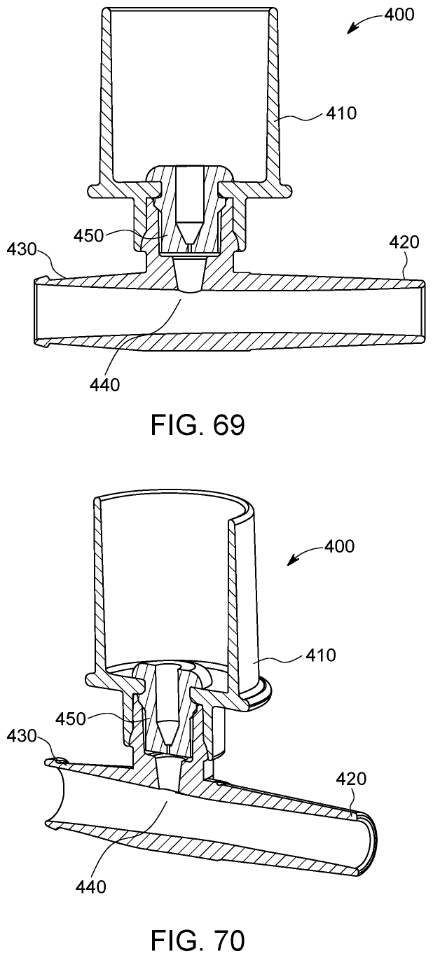

In some examples, as shown in FIGS. 69 and 70, the one-way valve 450 may be integrated into the fluid line connector 400. The one-way valve 450 may be configured to inhibit fluid flow from the junction region 440 through the first fluid evacuation line 23.

In the illustrated example, the one-way valve 450 is located within the connector body and is configured to inhibit fluid flow from the junction region 440 into and/or through the first inlet port 410. In use, the one-way valve is configured to be positioned upstream of the second inlet port 420 and above the outlet port 430.

In other examples, the one-way valve 450 may be spaced from the junction 440. For example, the one-way valve 450 may be provided separate from the fluid line connector 400 and spaced from the connector body 402. The one-way valve 450 may be in connection with the fluid evacuation line 23 and spaced from the connector body 402 by a connecting portion, such as a length of tubing. The connecting portion may connect the one-way valve 450 to the first inlet port 410. The one-way valve 450 may be configured to inhibit fluid flow from the body 402 through the first fluid evacuation line 23 past the location of the one-way valve 450.

The fluid line connector 400 may comprise at least one filter 425 in fluid connection with the first inlet port 410. The filter 425 comprises filter material. The filter 425 may include a filter housing, with the filter material located within the filter housing. The filter 425 (i.e. at least the filter material) may be positioned upstream of the one-way valve 450, as shown in FIG. 65, for example. The filter 425 may be configured to filter smoke and/or particulate matter contained in fluid flowing through the first fluid evacuation line 23. In some examples, the filter 425 may be positioned within the connector body 402. The filter 425 may be fixed to the connector body 402. In some examples, the filter 425 may be removably connectable to the connector body 402. The fluid line connector 400 may be configured such that, in use, the filter 425 is positioned upstream of the second inlet port 420 and/or upstream of the outlet port 430. The filter may be positioned upstream of the one-way valve 450.

It should be emphasized that many variations and modifications may be made to the examples described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

1-81. (canceled)

82. A medical instrument accessory for providing fluid to a body cavity of a patient during a surgical procedure, the medical instrument accessory comprising:
a body mountable over at least a portion of a shaft of a medical instrument, the body comprising:
an inner wall defining a lumen;
a proximal end;
a distal end defining an opening;
a fluid connecting portion configured to connect the lumen in fluid connection with a fluid source; and
a sealing element configured to provide a fluid-tight seal with the medical instrument;
wherein the sealing element is configured to allow rotation of the medical instrument accessory relative to the shaft of the medical instrument, such that rotational movement of the fluid connecting portion is decoupled from rotational movement of the shaft of the medical instrument.

83. The medical instrument accessory of claim 82, wherein the sealing element is configured to allow relative rotation of the fluid connecting portion about a longitudinal axis of the shaft of the medical instrument.

84. The medical instrument accessory of claim 82, wherein the fluid-tight seal is maintained during relative rotation between the shaft of the medical instrument and the medical instrument accessory.

85. The medical instrument accessory of claim 82, wherein the sealing element is configured to retain the medical instrument in position within the medical instrument accessory.

86. The medical instrument accessory of claim 82, wherein the sealing element is provided at or adjacent to a proximal end of the lumen and/or above the fluid connecting portion.

87. The medical instrument accessory of claim 82, further comprising at least one guide element on or within the inner wall.

88. The medical instrument accessory of claim 87, wherein the at least one guide element comprises a plurality of guide elements.

89. The medical instrument accessory of claim 87, wherein the at least one guide element is configured to position the shaft of the medical instrument within the lumen.

90. The medical instrument accessory of claim 89, wherein the at least one guide element holds the shaft of the medical instrument concentrically within the lumen.

91. The medical instrument accessory of claim 87, wherein the at least one guide element comprises one or more ribs extending inwardly from the inner wall.

92. The medical instrument accessory of claim 91, wherein the one or more ribs are located at or adjacent to the distal end of the body.

93. The medical instrument accessory of claim 82, wherein the sealing element is configured to provide a first resistance upon insertion of the medical instrument into the lumen, and a second resistance upon withdrawal of the medical instrument from the lumen.

94. The medical instrument accessory of claim 93, wherein the second resistance is greater than the first resistance.

95. The medical instrument accessory of claim 82, wherein the sealing element provides the fluid-tight seal with the shaft of the medical instrument.

96. The medical instrument accessory of claim 82, wherein the body further comprises a guide portion at the proximal end to guide the medical instrument into the lumen.

97. A medical instrument accessory for providing fluid to a body cavity of a patient during a surgical procedure, the medical instrument accessory comprising:
a body mountable over at least a portion of a shaft of a medical instrument, the body comprising:
an inner wall defining a lumen,
a proximal end;
a distal end defining an opening;
a first portion comprising a sealing element configured to provide a fluid-tight seal with the medical instrument; and
a second portion comprising a fluid connecting portion configured to connect the lumen in fluid connection with a fluid source,
wherein the second portion is rotatably coupled to the first portion.

98. A medical instrument accessory for providing fluid to and/or from a body cavity of a patient during a surgical procedure, the medical instrument accessory comprising:
a body mountable over at least a portion of a shaft of a medical instrument, the body comprising:
an inner wall defining a lumen,
a proximal end;
a distal end defining an opening;
a fluid connecting portion configured to connect the lumen in fluid communication with a fluid delivery tube;
a sealing element configured to provide a fluid-tight seal with the medical instrument; and
a securing element configured to maintain a position of the shaft of the medical instrument relative to at least a portion of the medical instrument accessory;
wherein the body is configured such that rotational movement of the fluid connecting portion is decoupled from rotational movement of the sealing element and/or the securing element.

99. The medical instrument accessory of claim 98, wherein the securing element is configured to maintain a position of the shaft of the medical instrument within the medical instrument accessory.

100. The medical instrument accessory of claim 98, wherein the securing element is the sealing element.

101. The medical instrument accessory of claim 98, wherein the body comprises:
a first conduit component; and
a second conduit component,
wherein the first conduit component comprises the sealing element and/or the securing element,
wherein the first conduit component and the second conduit component are configured to be connected together, and
wherein, when connected, the first conduit component and the second conduit component are configured to be rotatable relative to one another.
